(19) United States
(12) Patent Application Publication
(10) Pub. No.: US 2024/0341867 A1
(43) Pub. Date: Oct. 17, 2024

(54) OFFSCREEN INDICATOR VIEWER USER INTERFACE

(71) Applicant: Auris Health, Inc., Redwood City, CA (US)

(72) Inventors: Melissa STACHULSKI, Redwood City, CA (US); Fabien Y. SCHMITT, San Jose, CA (US)

(21) Appl. No.: 18/750,749

(22) Filed: Jun. 21, 2024

Related U.S. Application Data
(63) Continuation of application No. PCT/IB2022/062546, filed on Dec. 20, 2022.
(60) Provisional application No. 63/294,383, filed on Dec. 28, 2021.

(51) Int. Cl. A61B 34/00; A61B 34/35; A61B 34/37

(52) U.S. Cl. CPC A61B 34/25; A61B 34/35; A61B 34/37; A61B 34/70

(57) ABSTRACT

Surgical robotic systems may indicate locations of surgical tools. A surgical robotic system can include a robotic arm coupled to a surgical tool, a scope, and a viewer for displaying a field of view of a surgical site derived from the scope. The surgical robotic system may be configured to, in accordance with a determination that the surgical tool is not within the field of view, provide electrical signals for presenting a visual indicator on the viewer, indicating a location of the surgical tool.

### PRIORITY

This application is a continuation of International Patent Application No. PCT/IB2022/062546, filed Dec. 20, 2022, entitled “OFFSCREEN INDICATOR VIEWER USER INTERFACE,” which claims priority to U.S. Provisional Patent Application No. 63/294,383, entitled “OFFSCREEN INDICATOR VIEWER USER INTERFACE,” filed Dec. 28, 2021, the disclosures of each of which are incorporated by reference herein, in their entirety.

### TECHNICAL FIELD

The systems and methods disclosed herein are directed to devices and methods for indicating locations of surgical tools, and more particularly to surgical robotic systems for indicating locations of surgical tools.

### BACKGROUND

A robotically enabled medical system is capable of performing a variety of medical procedures, including both minimally invasive procedures, such as laparoscopy, and non-invasive procedures, such as endoscopy (e.g., bronchoscopy, ureteroscopy, gastroscopy, etc.).

Such robotic medical systems may include robotic arms configured to control the movement of surgical tool(s) during a given medical procedure. In order to achieve a desired pose of a surgical tool, a robotic arm may be placed into a particular pose during teleoperation. Some robotically enabled medical systems may include an arm support (e.g., a bar) that is connected to respective bases of the robotic arms and supports the robotic arms.

### SUMMARY

One or more instruments can be coupled to one or more robotic arms of a robotic medical system (e.g., surgical robotic system) for medical procedures. For example, an instrument can be coupled to a robotic arm as either a starting instrument to perform a procedure, or as a replacement instrument mid-procedure.

In a robotic system that includes multiple robotic arms, at least one robotic arm can be coupled to a camera or scope that provides a surgical field of view. When the medical instruments are within the surgical field of view, a user (e.g., a surgeon or physician assistant) can locate the instruments from the image of the surgical field of view. However, if an instrument is not within the surgical field of view, it is possible that movement of the instrument can lead to a contact between the instrument and an unintended tissue or organ. In addition, if the instrument is not within the surgical field of view, it may take additional time for the user to locate the instrument before starting to use the instrument, which can prolong the medical procedure.

Accordingly, there is a need for a robotic medical system that can facilitate the user to locate an instrument even when the instrument is positioned outside the surgical field of view.

As disclosed herein, a robotic medical system (e.g., a surgical robotic system) can include two or more robotic arms, such as a first robotic arm that is coupled to a surgical tool and a second robotic arm that is coupled to a scope (or a camera). The robotic medical system also includes a viewer for displaying a field of view of a surgical site. A user interface displayed on the viewer may be configured to include an off-screen indicator, which indicates a relative position of a surgical tool and/or a distance to the surgical tool so that the user can locate, and/or ascertain the distance to, the surgical tool.

As disclosed herein, the robotic medical system is configured to determine whether a tool is located within a field of view of the camera. In accordance with a determination that the tool is not within the field of view of the camera, the robotic medical system can provide to a physician and/or physician assistant an indication of a position of the tool and/or a distance to the tool from the field of view.

As disclosed herein, the robotic medical system has knowledge of the camera field of view (e.g., information indicating the camera field of view). In some embodiments, the field of view is determined based on an image provided by the camera. In some embodiments, the field of view is determined based on a position and an orientation of the camera (e.g., a predefined volume of space in front of the camera).

As disclosed herein, the robotic medical system is configured to allow movement of the robotic arms. In some embodiments, the robotic medical system may cause robotic movement of the robotic arms. In some embodiments, the robotic medical system may allow manual movement of the robotic arms.

As disclosed the robotic medical system is configured to determine the position and orientation of the robotic arms, and more specifically, their associated tools. In some embodiments, the robotic medical system determines that at least one of the robotic arms has moved (and hence the position of an associated tool has changed) and updates the off-screen indicator to indicate the changed position of the associated tool. In some embodiments, the robotic medical system updates the off-screen indicator in real time (e.g., while the associated tool is moving) to indicate the changed position of the associated tool.

Accordingly, the systems and/or methods disclosed herein advantageously improve the operation of robotic medical systems during surgery. For example, a user can be notified of a tool position and distance even when the tool is located outside the field of view of a camera. This leads to a better overall user experience because the user can determine the position of the tool quickly. The ability to determine the position of the tool when the tool is located outside the field of view may also improve the safety of the surgery.

The systems, methods and devices of this disclosure each have several innovative aspects, no single one of which is solely responsible for the desirable attributes disclosed herein.

In accordance with some embodiments of the present disclosure, a robotic system includes a first robotic arm coupled to a first surgical tool; a second robotic arm coupled to a second surgical tool; and a third robotic arm coupled to a scope. The robotic system also includes a viewer for displaying a field of view of a surgical site derived from the scope. The robotic system further includes one or more processors, and memory storing instructions for execution by the one or more processors. The stored instructions include instructions for, in accordance with a determination that the second surgical tool is within the field of view and the first surgical tool is not within the field of view, providing electrical signals for presenting a first visual indicator on the viewer. The first visual indicator indicates a location of the first surgical tool.

In some embodiments, the stored instructions also include instructions for: providing electrical signals for presenting the first visual indicator at a first location on the viewer to indicate the location of the first surgical tool; and, subsequent to providing the electrical signals for presenting the first visual indicator at the first location on the viewer, in accordance with a determination that the location of the first surgical tool has changed, providing electrical signals for presenting the first visual indicator at a second location, on the viewer, that corresponds to the changed location of the first surgical tool.

In some embodiments, the first visual indicator also indicates a distance to the first surgical tool from the field of view.

In some embodiments, the stored instructions also include instructions for, subsequent to providing the electrical signals for presenting the first visual indicator on the viewer, in accordance with a determination that the distance from the field of view to the first surgical tool has changed, providing electrical signals for presenting an updated first visual indicator on the viewer that indicates the changed distance from the field of view to the first surgical tool.

In some embodiments, the stored instructions also include instructions for, subsequent to providing the electrical signals for presenting the first visual indicator on the viewer, in accordance with a determination that the first surgical tool is within the field of view, ceasing to provide the electrical signals for presenting the first visual indicator on the viewer.

In some embodiments, the first visual indicator includes a graphical element having a size that indicates the distance to the first surgical tool from the field of view.

In some embodiments, the first visual indicator includes information identifying a robotic arm associated with the graphical element.

In some embodiments, the first visual indicator identifies a type of the first surgical tool.

In some embodiments, the first visual indicator includes a graphical representation of the first surgical tool.

In some embodiments, the first visual indicator is concurrently displayed with a display of the field of view of the surgical site on the viewer.

In some embodiments, the first visual indicator is displayed around the display of the field of view of the surgical site.

In some embodiments, the first visual indicator is displayed over the display of the field of view of the surgical site.

In some embodiments, the stored instructions also include instructions for providing electrical signals for highlighting a predefined region of the display of the field of view of the surgical site and the first visual indicator is displayed along a periphery of the predefined region.

In some embodiments, the stored instructions also include instructions for providing a rendered image that includes the first visual indicator.

In some embodiments, the rendered image also includes a second visual indicator indicating a location of the second surgical tool.

In some embodiments, the first visual indicator includes information identifying the first robotic arm associated with the first surgical tool and the second visual indicator includes information identifying the second robotic arm associated with the second surgical tool.

In some embodiments, the first visual indicator includes a graphical representation of the first surgical tool and the second visual indicator includes a graphical representation of the second surgical tool.

In some embodiments, the stored instructions include instructions for replacing the display of the field of view with the rendered image.

In some embodiments, the stored instructions include instructions for overlaying the rendered image partially over the display of the field of view.

In some embodiments, the stored instructions include instructions for, in response to receiving a request for displaying the first visual indicator and in accordance with the determination that second surgical tool is within the field of view and the first surgical tool is not within the field of view, providing the electrical signals for presenting the first visual indicator on the viewer.

In some embodiments, the stored instructions include instructions for, in response to determining that the request for displaying the first visual indicator has ceased, ceasing to provide the electrical signals for presenting the first visual indicator on the viewer.

In some embodiments, the first robotic arm and the second robotic arm are positioned bilaterally relative to an operating table.

In some embodiments, the first robotic arm is supported on a first adjustable arm support and the second robotic arm is supported on a second adjustable arm support.

In some embodiments, the first robotic arm and the second robotic arm are positioned on a same side of an operating table.

In some embodiments, the first robotic arm and the second robotic arm are supported on an adjustable arm support.

In some embodiments, the viewer is part of a surgeon console.

In some embodiments, the surgeon console includes an input device. The stored instructions include instructions for: receiving an input on the input device; and in response to receiving the input on the input device, providing electrical signals for presenting a bird's eye view of the first robotic arm, the second robotic arm, and the third robotic arm.

In some embodiments, the input device includes a foot pedal.

In accordance with some embodiments, an electronic device is in communication with a robotic system with a first robotic arm coupled to a first surgical tool, a second robotic arm coupled to a second surgical tool, a scope, and a viewer. The electronic device includes one or more processors and memory storing instructions for execution by the one or more processors. The stored instructions include instructions for, in accordance with a determination that the second surgical tool is within a field of view and the first surgical tool is not within the field of view, providing electrical signals for presenting a first visual indicator on the viewer. The first visual indicator indicates a location of the first surgical tool.

In accordance with some embodiments, a computer-readable storage medium stores instructions for execution by one or more processors in communication with a robotic system with a first robotic arm coupled to a first surgical tool, a second robotic arm coupled to a second surgical tool, a scope, and a viewer. The stored instructions include instructions for, in accordance with a determination that the second surgical tool is within a field of view and the first surgical tool is not within the field of view, providing electrical signals for presenting a first visual indicator on the viewer. The first visual indicator indicates a location of the first surgical tool.

In accordance with some embodiments, a robotic system includes a first robotic arm coupled to a first surgical tool; a second robotic arm coupled to a second surgical tool; a scope; and a viewer for displaying a field of view of a surgical site derived from the scope. The robotic system also includes one or more processors and memory storing instructions for execution by the one or more processors. The stored instructions include instructions for, in response to receiving a request for displaying a first visual indicator, providing electrical signals for presenting the first visual indicator on the viewer. The first visual indicator indicates a location of the first surgical tool.

In accordance with some embodiments, a robotic system includes a first robotic arm coupled to a first surgical tool; a second robotic arm coupled to a second surgical tool; a scope; and a viewer for displaying a field of view of a surgical site derived from the scope. The robotic system also includes one or more processors and memory storing instructions for execution by the one or more processors. The stored instructions include instructions for providing electrical signals for presenting a first visual indicator on the viewer. The first visual indicator indicates a location of the first surgical tool.

In accordance with some embodiments, a robotic system includes a first robotic arm coupled to a first surgical tool; a scope; and a viewer for displaying a field of view of a surgical site derived from the scope. The robotic system also includes one or more processors and memory storing instructions for execution by the one or more processors. The stored instructions including instructions for, in response to receiving a request for displaying a first visual indicator, providing electrical signals for presenting the first visual indicator on the viewer. The first visual indicator indicates a location of the first surgical tool.

In accordance with some embodiments, a robotic system includes a first robotic arm coupled to a first surgical tool; a scope; and a viewer for displaying a field of view of a surgical site derived from the scope. The robotic system also includes one or more processors and memory storing instructions for execution by the one or more processors. The stored instructions include instructions for, providing electrical signals for presenting a first visual indicator on the viewer. The first visual indicator indicates a location of the first surgical tool.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s).

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopy procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopy procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance with an example embodiment.

FIG. 21 illustrates an exemplary robotic system according to some embodiments.

FIG. 22 illustrates another view of an exemplary robotic system according to some embodiments.

FIG. 23 illustrates components of a robotic medical system in accordance with some embodiments.

FIGS. 24A to 24C illustrate different views of an exemplary robotic arm according to some embodiments.

FIG. 25 illustrates a perspective view of a robotic medical system in accordance with some embodiments.

FIGS. 26A and 26B illustrate a camera field of view in a three-dimensional space in accordance with some embodiments.

FIGS. 27A to 27J illustrate exemplary user interfaces displayed on a display device in accordance with some embodiments.

FIGS. 28A to 28D illustrate exemplary user interfaces displayed on a display device in accordance with some embodiments.

FIGS. 29A to 29C illustrate a flowchart diagram for a method performed by one or more processors of a surgical robotic system, in accordance with some embodiments.

FIG. 30 is a schematic diagram illustrating electronic components of a robotic medical system in accordance with some embodiments.

### DETAILED DESCRIPTION

### 1. Overview.

Aspects of the present disclosure may be integrated into a robotically enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other embodiments of the disclosed concepts are possible, and various advantages can be achieved with the disclosed embodiments. Headings are included herein for reference and to aid in locating various sections. These headings are not intended to limit the scope of the concepts described with respect thereto. Such concepts may have applicability throughout the entire specification.

### A. Robotic System—Cart.

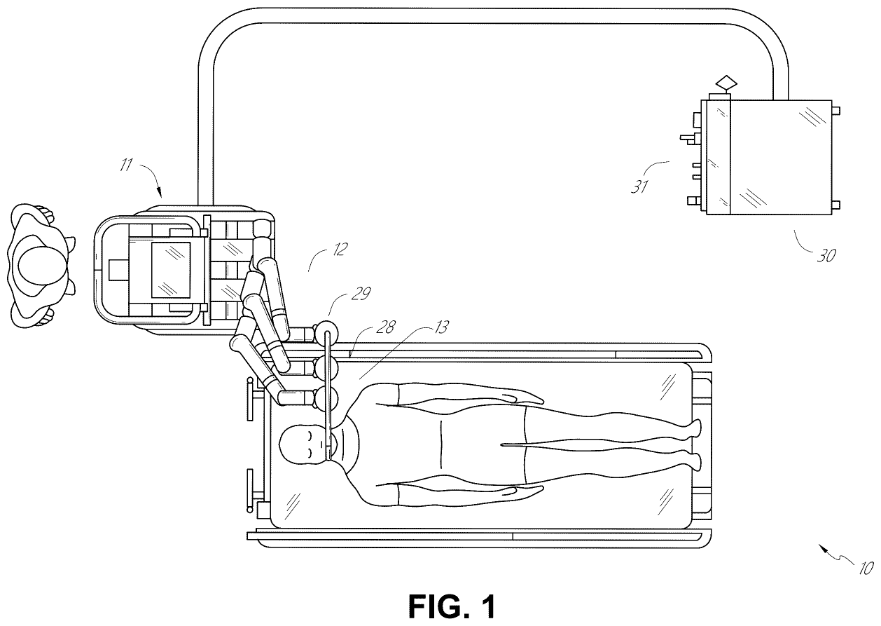

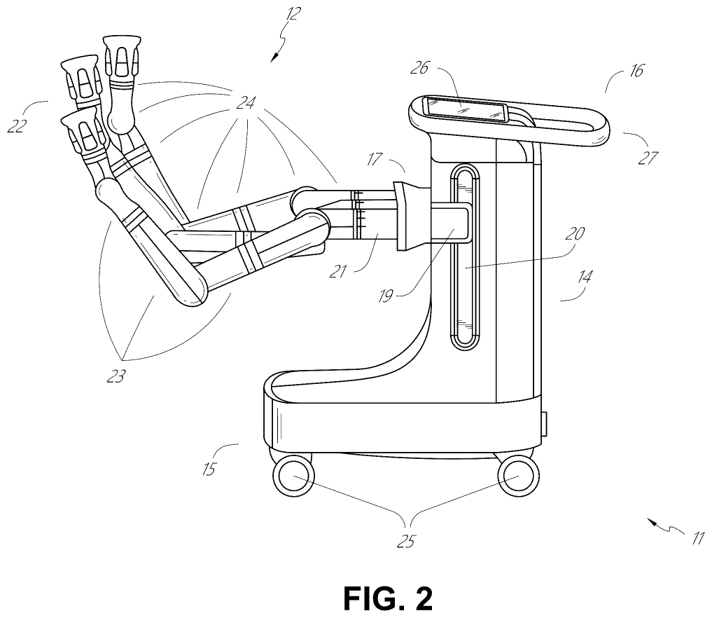

The robotically enabled medical system may be configured in a variety of ways depending on the particular procedure. FIG. 1 illustrates an embodiment of a cart-based robotically enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart, the support for controls, optics, fluidics, and/or navigation may be provided through a separate cable.

FIG. 2 provides a detailed illustration of an embodiment of the cart from the cart-based robotically enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. Each of the arms 12 have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes rollable wheel-shaped casters 25 that allow for the cart to easily move around the room prior to a procedure. After reaching the appropriate position, the casters 25 may be immobilized using wheel locks to hold the cart 11 in place during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position, the physician may view the console 16, robotic arms 12, and patient while operating the console 16 from behind the cart 11. As shown, the console 16 also includes a handle 27 to assist with maneuvering and stabilizing cart 11.

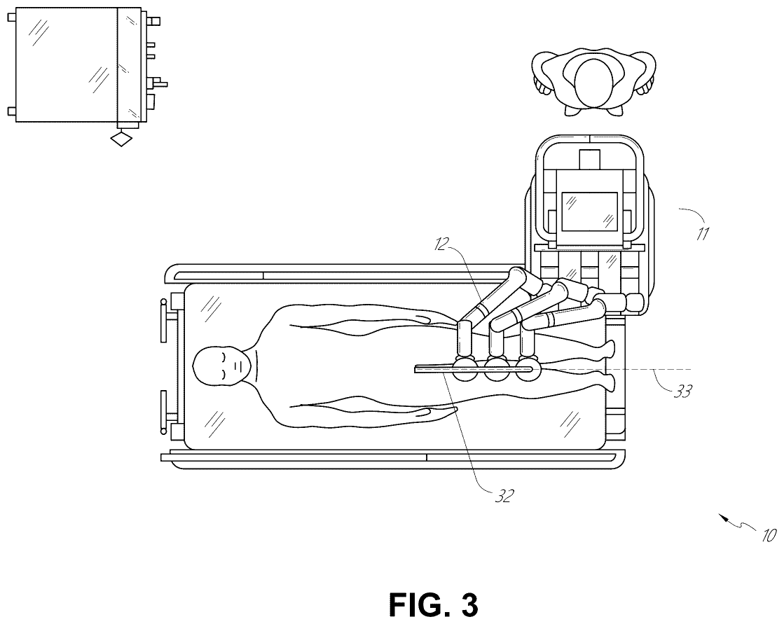

FIG. 3 illustrates an embodiment of a robotically enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

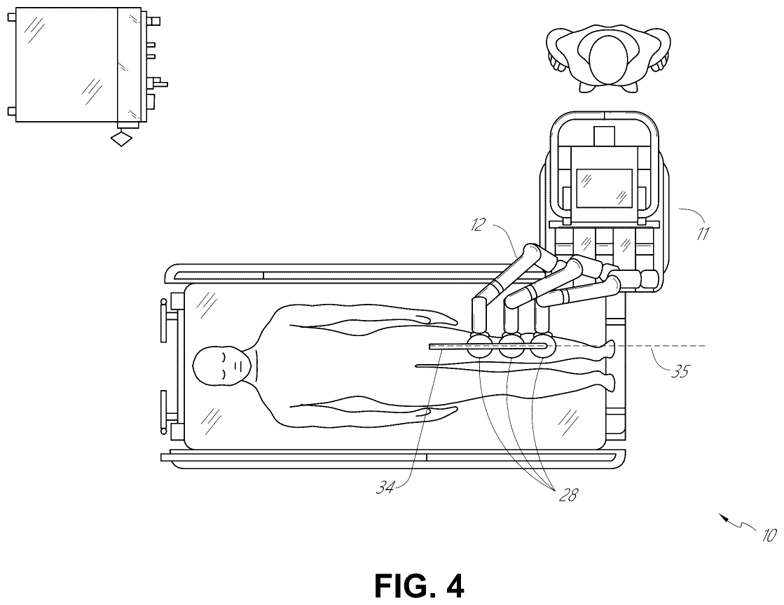

FIG. 4 illustrates an embodiment of a robotically enabled system similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

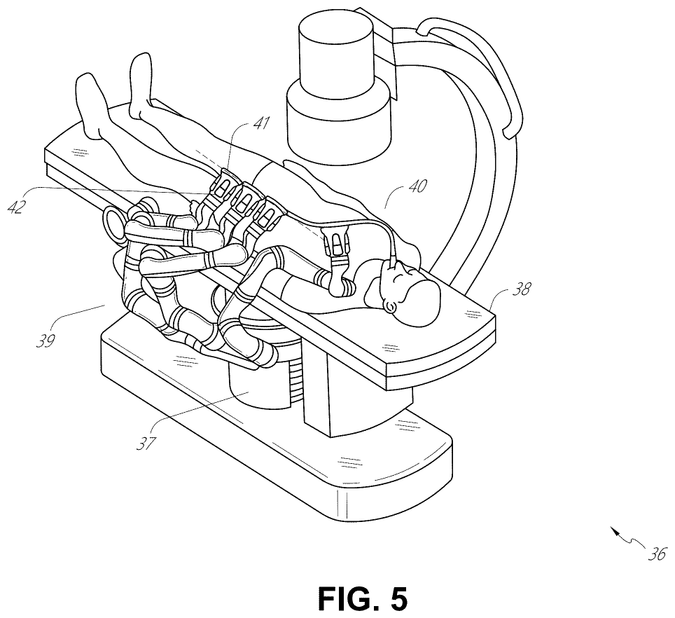

Embodiments of the robotically enabled medical system may also incorporate the patient's table. Incorporation of the table reduces the amount of capital equipment within the operating room by removing the cart, which allows greater access to the patient. FIG. 5 illustrates an embodiment of such a robotically enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around table 38.

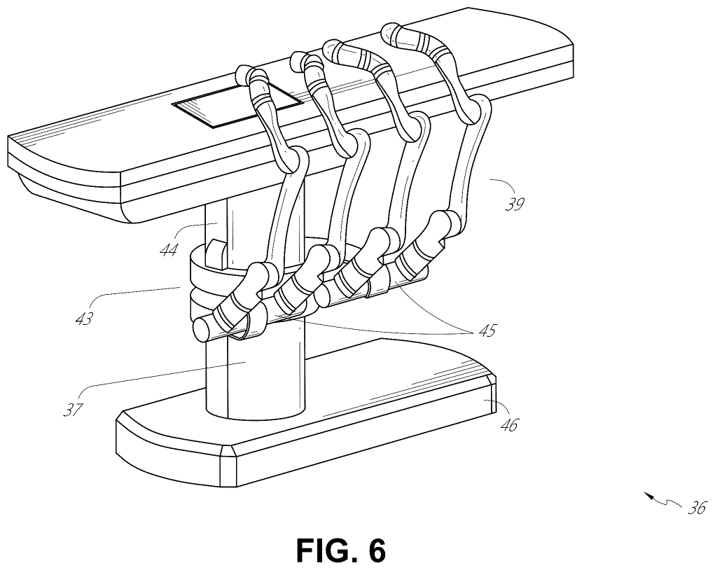

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independent of the other carriages. While carriages 43 need not surround the column 37 or even be circular, the ring-shape as shown facilitates rotation of the carriages 43 around the column 37 while maintaining structural balance. Rotation and translation of the carriages 43 allows the system to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

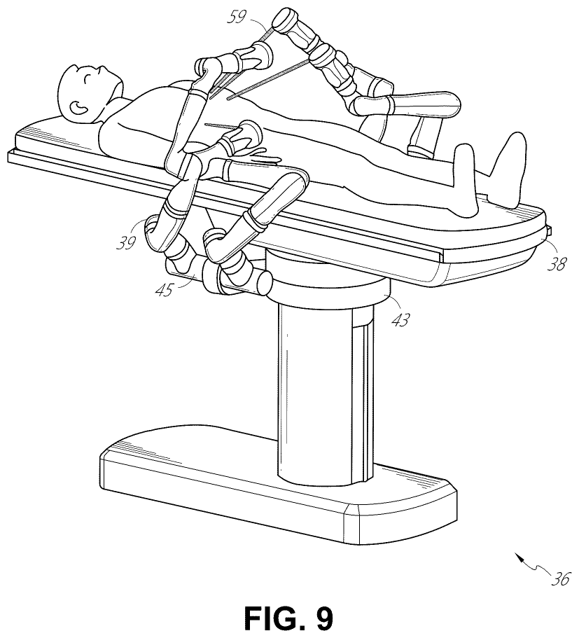

The arms 39 may be mounted on the carriages through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of table 38 (as shown in FIG. 6), on opposite sides of table 38 (as shown in FIG. 9), or on adjacent sides of the table 38 (not shown).

The column 37 structurally provides support for the table 38, and a path for vertical translation of the carriages. Internally, the column 37 may be equipped with lead screws for guiding vertical translation of the carriages, and motors to mechanize the translation of said carriages based the lead screws. The column 37 may also convey power and control signals to the carriage 43 and robotic arms 39 mounted thereon.

The table base 46 serves a similar function as the cart base 15 in cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

Continuing with FIG. 6, the system 36 may also include a tower (not shown) that divides the functionality of system 36 between table and tower to reduce the form factor and bulk of the table. As in earlier disclosed embodiments, the tower may provide a variety of support functionalities to table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base for potential stowage of the robotic arms. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

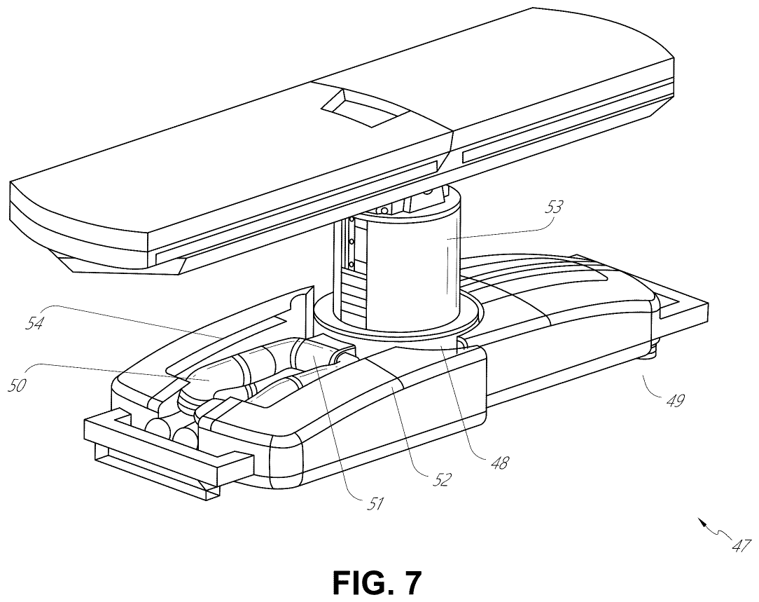

In some embodiments, a table base may stow and store the robotic arms when not in use. FIG. 7 illustrates a system 47 that stows robotic arms in an embodiment of the table-based system. In system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use. The base covers 52 may be sealed with a membrane 54 along the edges of its opening to prevent dirt and fluid ingress when closed.

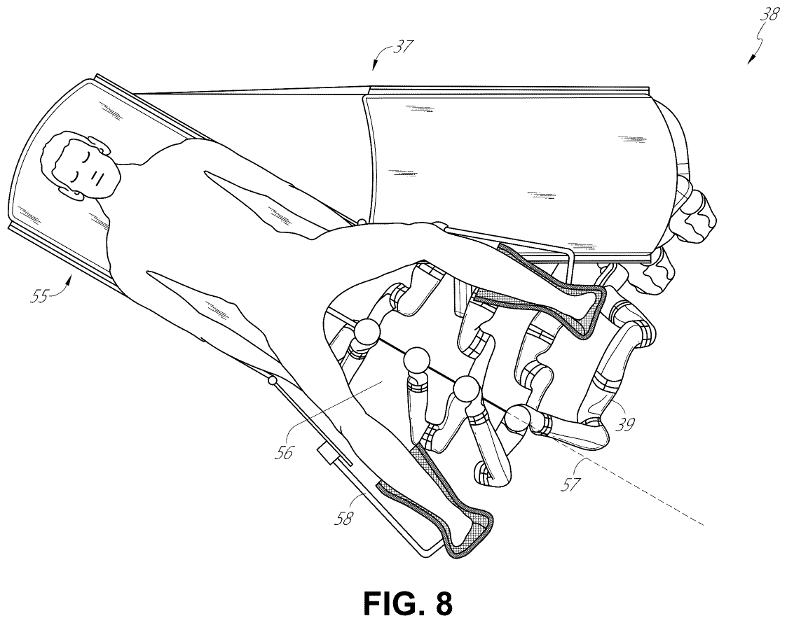

FIG. 8 illustrates an embodiment of a robotically enabled table-based system configured for a ureteroscopy procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

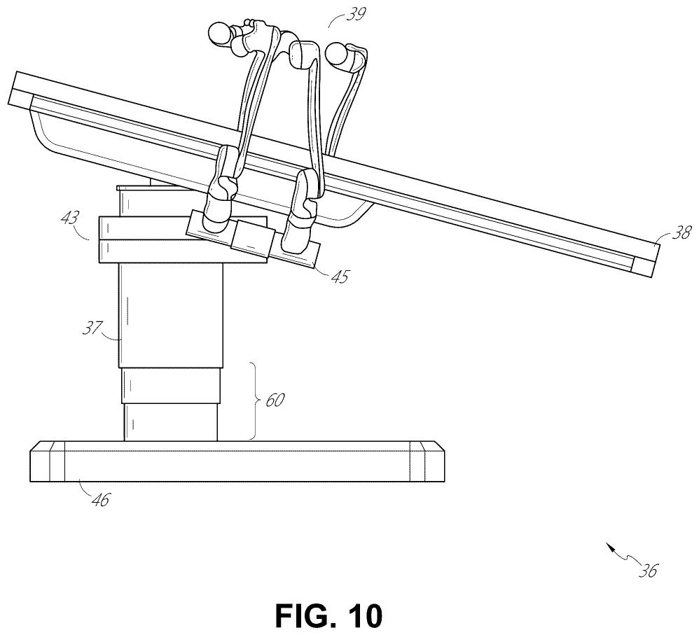

To accommodate laparoscopic procedures, the robotically enabled table system may also tilt the platform to a desired angle. FIG. 10 illustrates an embodiment of the robotically enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the arms 39 maintain the same planar relationship with table 38. To accommodate steeper angles, the column 37 may also include telescoping portions 60 that allow vertical extension of column 37 to keep the table 38 from touching the floor or colliding with base 46.

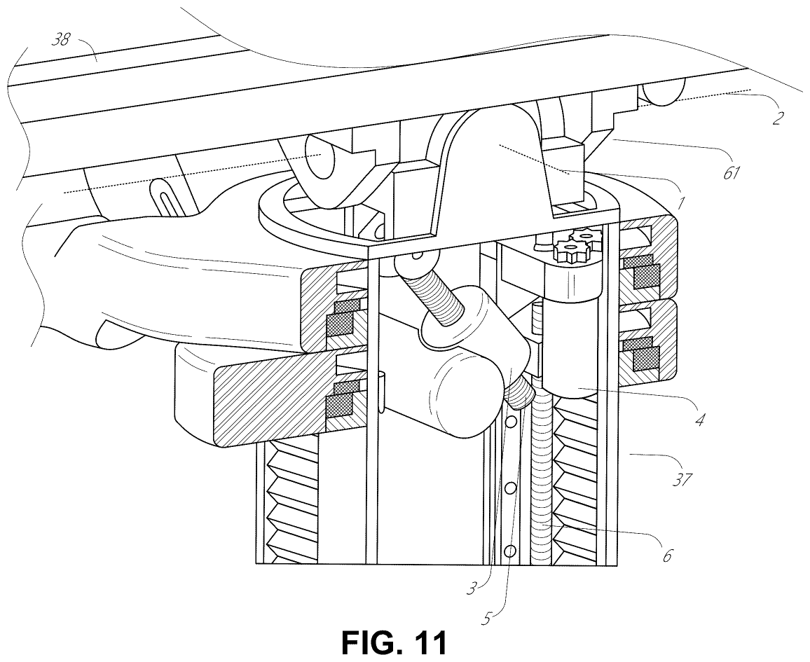

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

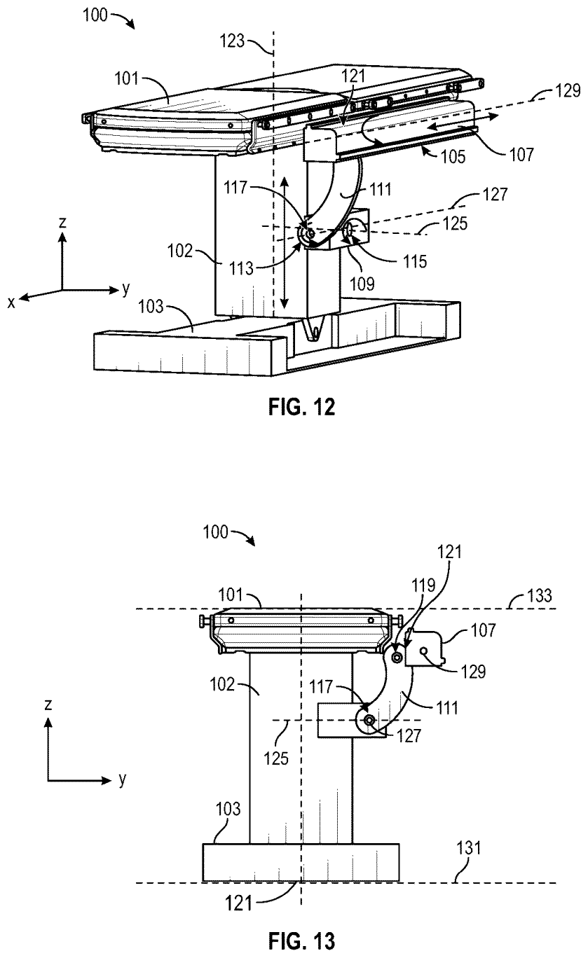

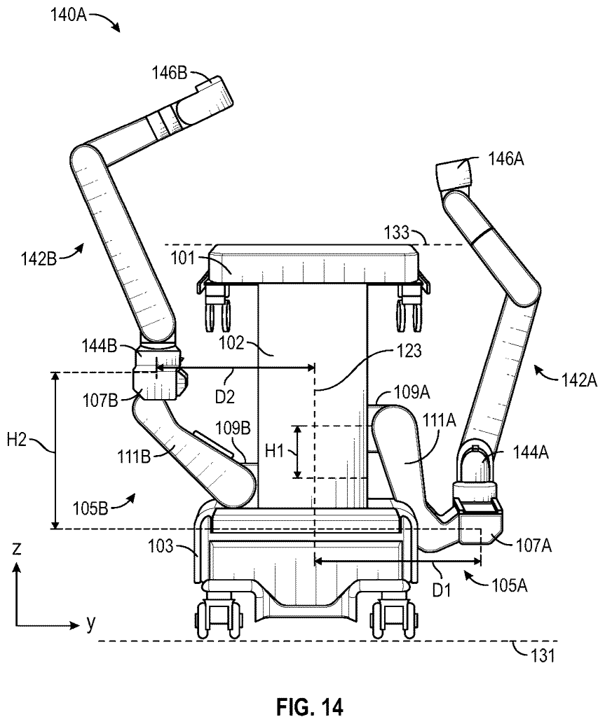

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporate electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

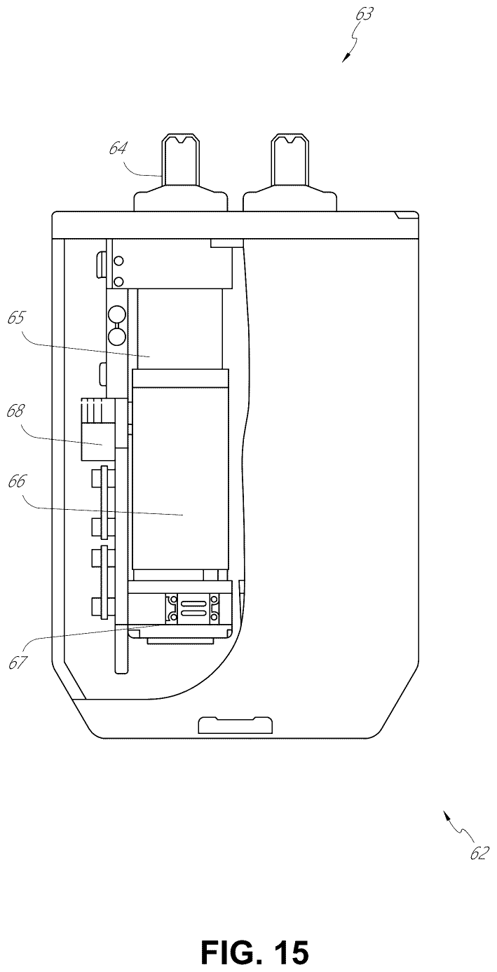

FIG. 15 illustrates an example instrument driver. Positioned at the distal end of a robotic arm, instrument driver 62 comprises of one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independent controlled and motorized, the instrument driver 62 may provide multiple (four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

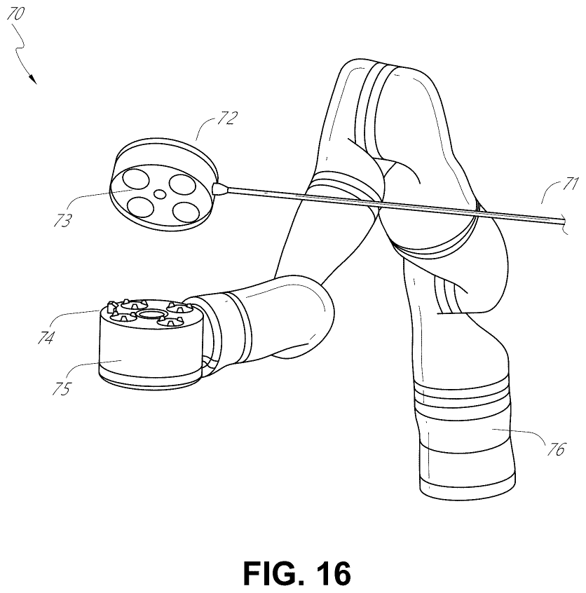

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise of a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include of an optical camera. The shaft 71 may also accommodate optical fibers to carry light from proximally located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft during an endoscopic procedure.

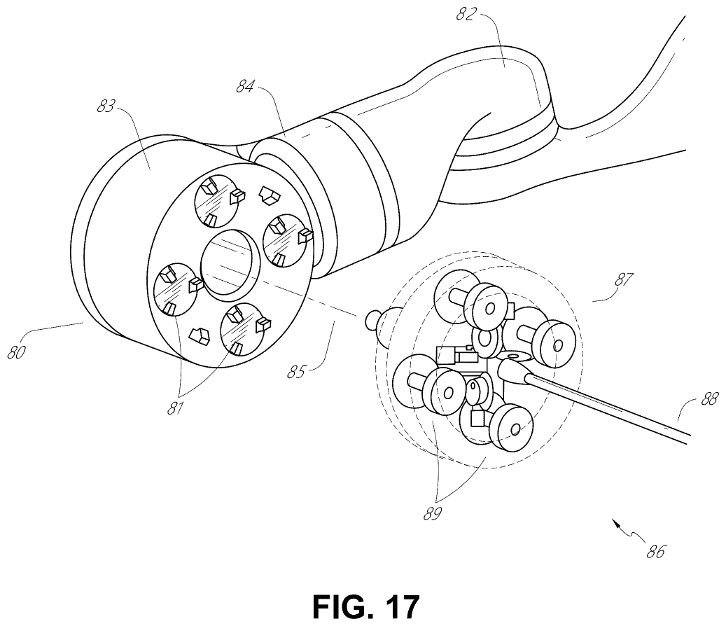

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts and may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, instrument shaft 88 extends from the center of instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

FIG. 18 illustrates an instrument having an instrument-based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

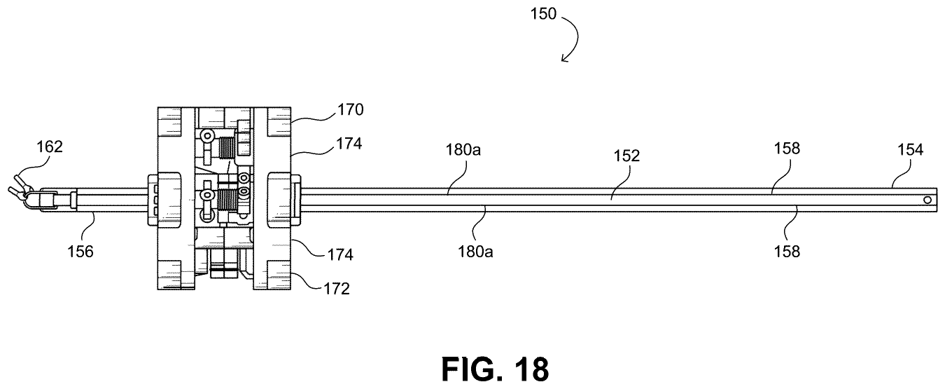

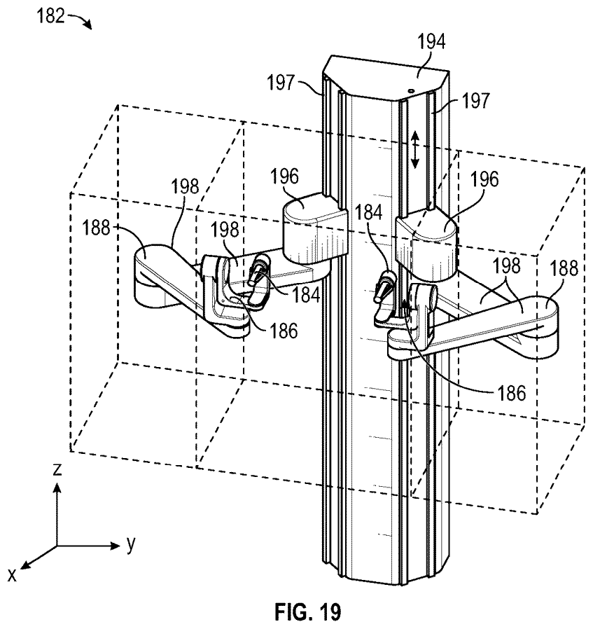

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

FIG. 20 is a block diagram illustrating a localization system 90 that estimates a location of one or more elements of the robotic system, such as the location of the instrument, in accordance with an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans are reconstructed into three-dimensional images, which are visualized, e.g., as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images and are particularly appropriate for bronchoscopy.

In some embodiments, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter) may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Off-Screen Indicator.

This application discloses robotic medical systems that determine whether a tool is located outside a surgical field of view provided by a camera. Based on this data, the system can provide an indication of the position of the tool.

In some embodiments, the robotic medical system can provide the indication via a user interface or display device of the robotic medical system. For example, in some embodiments, the robotic medical system provides a visual indication of a position (or a relative direction) of the tool.

In some embodiments, the robotic medical system updates the visual indication in response to determining that the tool has changed its position (e.g., the tool has changed its relative direction to the field of view or its distance to the field of view).

In some embodiments, the visual indication is provided only when at least one tool is located outside the field of view. In some embodiments, the visual indication is provided regardless of whether any tool is located outside the field of view. In some embodiments, the robotic medical system provides the visual indication in response to (or while) receiving a request to provide the visual indication. In some embodiments, the robotic medical system ceases to provide the visual indication in response to determining that the request to provide the visual indication has ceased or in response to receiving a request to cease display of the visual indication.

### A. Robotic System.

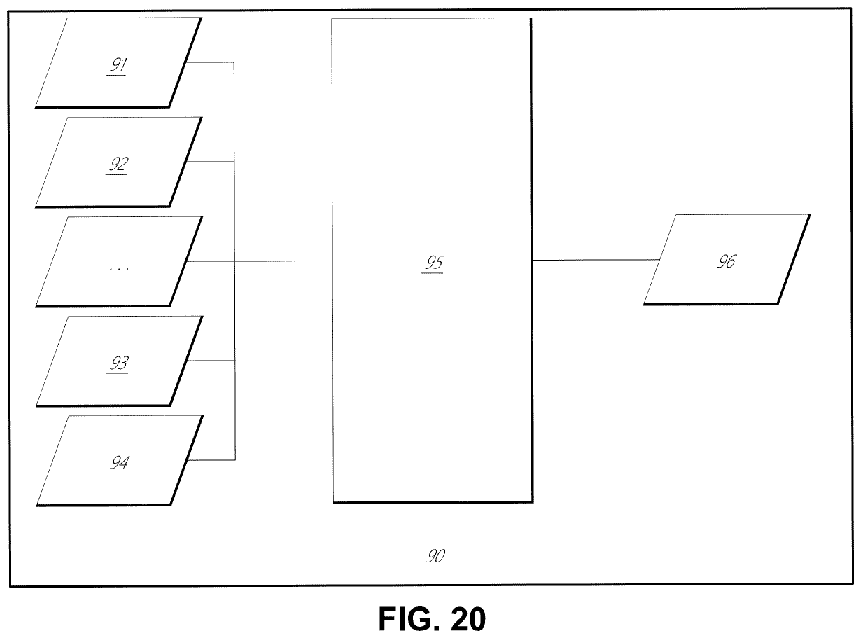

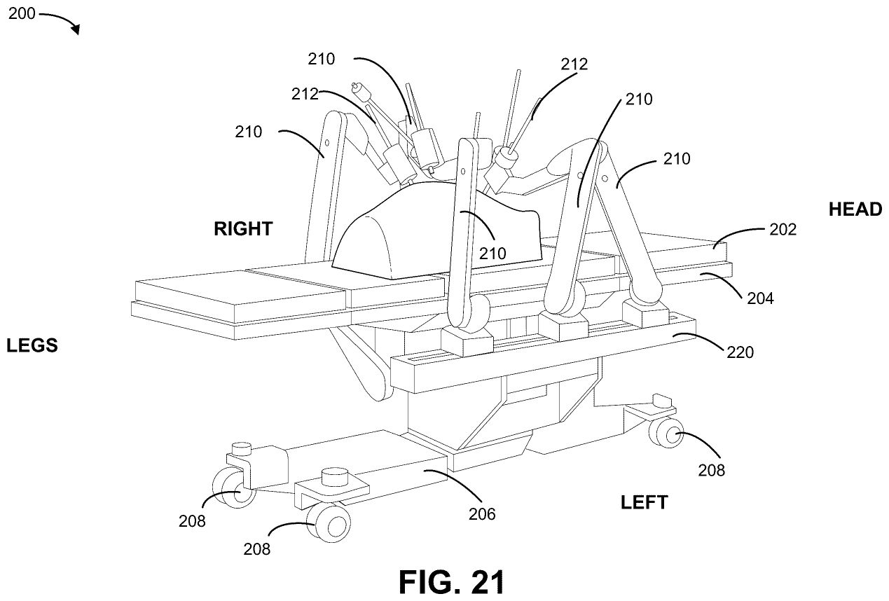

FIG. 21 illustrates an exemplary robotic medical system 200 according to some embodiments. In some embodiments, the robotic medical system 200 is a robotic surgery system. In the example of FIG. 21, the robotic medical system 200 comprises a patient support platform 202 (e.g., a patient platform, a table, a bed, etc.). The two ends along the length of the patient support platform 202 are respectively referred to as “head” and “leg”. The two sides of the patient support platform 202 are respectively referred to as “left” and “right.” The patient support platform 202 includes a support 204 (e.g., a rigid frame) for the patient support platform 202.

The robotic medical system 200 also comprises a base 206 for supporting the robotic medical system 200. The base 206 includes wheels 208 that allow the robotic medical system 200 to be easily movable or repositionable in a physical environment. In some embodiments, the wheels 208 are omitted from the robotic medical system 200 or are retractable, and the base 206 can rest directly on the ground or floor. In some embodiments, the wheels 208 are replaced with feet.

The robotic medical system 200 includes one or more robotic arms 210. The robotic arms 210 can be configured to perform robotic medical procedures as described above with reference to FIGS. 1-20. Although FIG. 21 shows five robotic arms 210, it should be appreciated that the robotic medical system 200 may include any number of robotic arms, including less than five or six or more.

The robotic medical system 200 also includes one or more bars 220 (e.g., adjustable arm support or an adjustable bar) that support the robotic arms 210. Each of the robotic arms 210 is supported on, and movably coupled to, a bar 220, by a respective base joint of the robotic arm. In some embodiments, and as described in FIG. 12, bar 220 can provide several degrees of freedom, including lift, lateral translation, tilt, etc. In some embodiments, each of the robotic arms 210 and/or the adjustable arm supports 220 is also referred to as a respective kinematic chain.

FIG. 21 shows three robotic arms 210 supported by the bar 220 that is in the field of view of the figure. The two remaining robotic arms are supported by another bar that is located across the other length of the patient support platform 202.

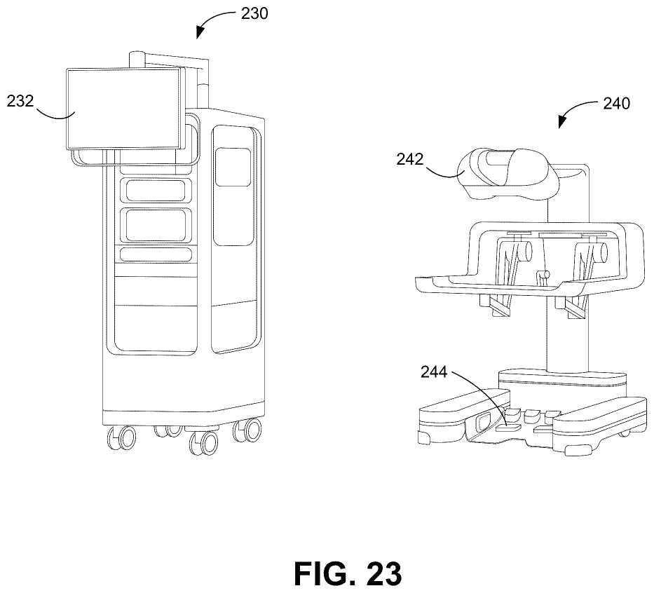

In some embodiments, the adjustable arm supports 220 can be configured to provide a base position for one or more of the robotic arms 210 for a robotic medical procedure. A robotic arm 210 can be positioned relative to the patient support platform 202 by translating the robotic arm 210 along a length of its underlying bar 220 and/or by adjusting a position and/or orientation of the robotic arm 210 via one or more joints and/or links (see, e.g., FIG. 23). In some embodiments, the bar pose can be changed via manual manipulation, teleoperation, and/or power assisted motion.

In some embodiments, the adjustable arm support 220 can be translated along a length of the patient support platform 202. In some embodiments, translation of the bar 220 along a length of the patient support platform 202 causes one or more of the robotic arms 210 supported by the bar 220 to be simultaneously translated with the bar or relative to the bar. In some embodiments, the bar 220 can be translated while keeping one or more of the robotic arms stationary with respect to the base 206 of the robotic medical system 200.

In the example of FIG. 21, the adjustable arm support 220 is located along a length of the patient support platform 202. In some embodiments, the adjustable arm support 220 may extend across a partial or full length of the patient support platform 202, and/or across a partial or full width of the patient support platform 202.

During a robotic medical procedure, one or more of the robotic arms 210 can also be configured to hold instruments 212 (e.g., robotically controlled medical instruments or tools, such as an endoscope and/or any other instruments (e.g., sensors, illumination instrument, cutting instrument, etc.) that may be used during surgery), and/or be coupled to one or more accessories, including one or more cannulas, in accordance with some embodiments.

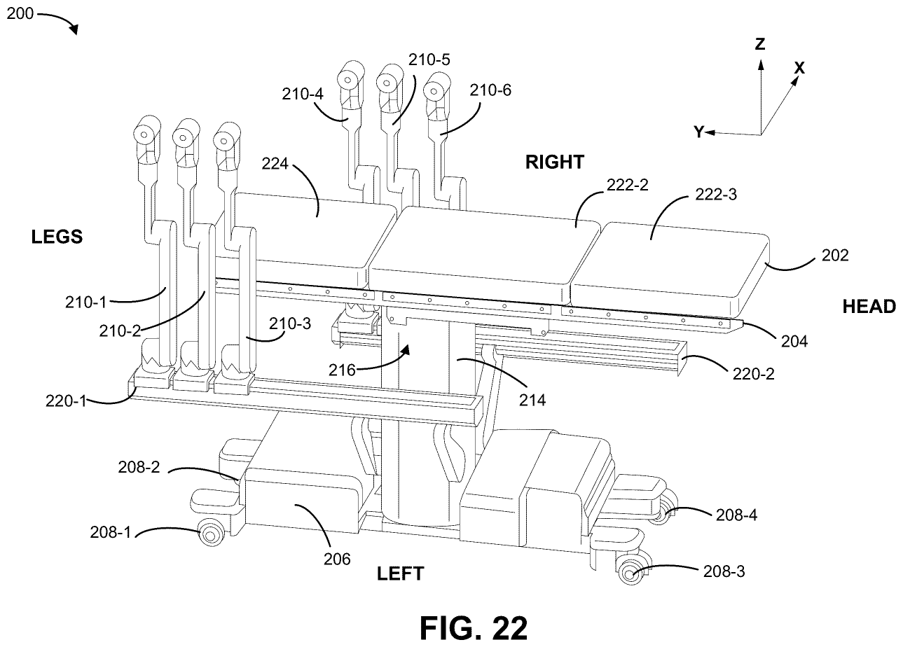

FIG. 22 illustrates another view of the exemplary robotic medical system 200 in FIG. 21 according to some embodiments. In this example, the robotic medical system 200 includes six robotic arms 210-1, 210-2, 210-3, 210-4, 210-5, and 210-6. The patient platform 202 is supported by a column 214 that extends between the base 206 and the patient platform 202. In some embodiments, the patient platform 202 comprises a tilt mechanism 216. The tilt mechanism 216 can be positioned between the column 214 and the patient platform 202 to allow the patient platform 202 to pivot, rotate, or tilt relative to the column 214. The tilt mechanism 216 can be configured to allow for lateral and/or longitudinal tilt of the patient platform 202. In some embodiments, the tilt mechanism 216 allows for simultaneous lateral and longitudinal tilt of the patient platform 202.

FIG. 22 shows the patient platform 202 in an untilted state or position. In some embodiments, the untilted state or position is a default position of the patient platform 202. In some embodiments, the default position of the patient platform 202 is a substantially horizontal position as shown in FIG. 22. As illustrated, in the untilted state, the patient platform 202 can be positioned horizontally or parallel to a surface that supports the robotic medical system 200 (e.g., the ground or floor). In some embodiments, the term “untilted” refers to a state in which the angle between the default position and the current position is less than a threshold angle (e.g., less than 5 degrees, or less than an angle that would cause the patient to shift on the patient platform, etc.). In some embodiments, the term “untilted” refers to a state in which the patient platform is substantially perpendicular to the direction of gravity, irrespective of the angle formed by the surface that supports the robotic medical system relative to gravity.

With continued reference to FIG. 22, in the illustrated example of the robotic medical system 200, the patient platform 202 includes a support 204. In some embodiments, the support 204 includes a rigid support structure or frame, and can support one or more surfaces, pads, or cushions 222. An upper surface of the patient platform 202 can include a support surface 224. During a medical procedure, a patient can be placed on the support surface 224.

FIG. 22 shows the robotic arms 210 and the adjustable arm supports 220 in an exemplary deployed configuration in which the robotic arms 210 reach above the patient platform 202. In some embodiments, due to the configuration of the robotic medical system 200, which enables stowage of different components beneath the patient platform 202, the robotic arms 210 and the arm supports 220 can occupy a space underneath the patient platform 202. Thus, in some embodiments, the tilt mechanism 216 has a low-profile and/or low volume in order to increase the space available for storage below.

FIG. 22 also illustrates an example, x, y, and z coordinate system that may be used to describe certain features of the embodiments disclosed herein. It will be appreciated that this coordinate system is provided for purposes of example and explanation only and that other coordinate systems may be used. In the illustrated example, the x-direction or x-axis extends in a lateral direction across the patient platform 202 when the patient platform 202 is in an untilted state. In some configurations, the x-direction extends across the patient platform 202 from one lateral side (e.g., the right side) to the other lateral side (e.g., the left side) when the patient platform 202 is in an untilted state. The y-direction or y-axis extends in a longitudinal direction along the patient platform 202 when the patient platform 202 is in an untilted state. That is, the y-direction extends along the patient platform 202 from one longitudinal end (e.g., the head end) to the other longitudinal end (e.g., the legs end) when the patient platform 202 is in an untilted state. In an untilted state, the patient platform 202 can lie in or be parallel to the x-y plane, which can be parallel to the floor or ground. In the illustrated example, the z-direction or z-axis extends along the column 214 in a vertical direction. In some embodiments, the tilt mechanism 216 is configured to laterally tilt the patient platform 202 by rotating the patient platform 202 about a lateral tilt axis that is parallel to the y-axis. The tilt mechanism 216 can further be configured to longitudinally tilt the patient platform 202 by rotating the patient platform 202 about a longitudinal tilt axis that is parallel to the x-axis.

In some embodiments, the robotic medical system 200 includes a tower 230 (e.g., tower viewer) or a physician console 240 (or both), as illustrated in FIG. 23. The tower 230 may correspond to the tower 30 described above, and may provide support for controls, electronics, fluidics, optics, sensors, and/or power for the patient support platform 202 and the physician console 240. In some embodiments, the tower 230 includes a display device 232. The display device 232 can include a user interface for displaying a surgical view obtained by one or more cameras 606 of the robotic medical system and/or one or more notifications to an operator of the robotic medical system 200. In some embodiments, the physician console 240 can include a display device 242 having a user interface used by the physician operator for operating the patient support platform 202. For example, the display device 242 may include a user interface for displaying a surgical view obtained by one or more cameras 606 of the robotic medical system and/or one or more notifications to an operator of the robotic medical system 200. The physician console 240 can provide both robotic controls and pre-operative and real-time information of a medical procedure to a physician operator. In some embodiments, the physician console 240 includes one or more input devices (e.g., buttons, switches, touch-sensitive surfaces, gimbals, etc.), such as a foot pedal 244.

### B. Robotic Arm.

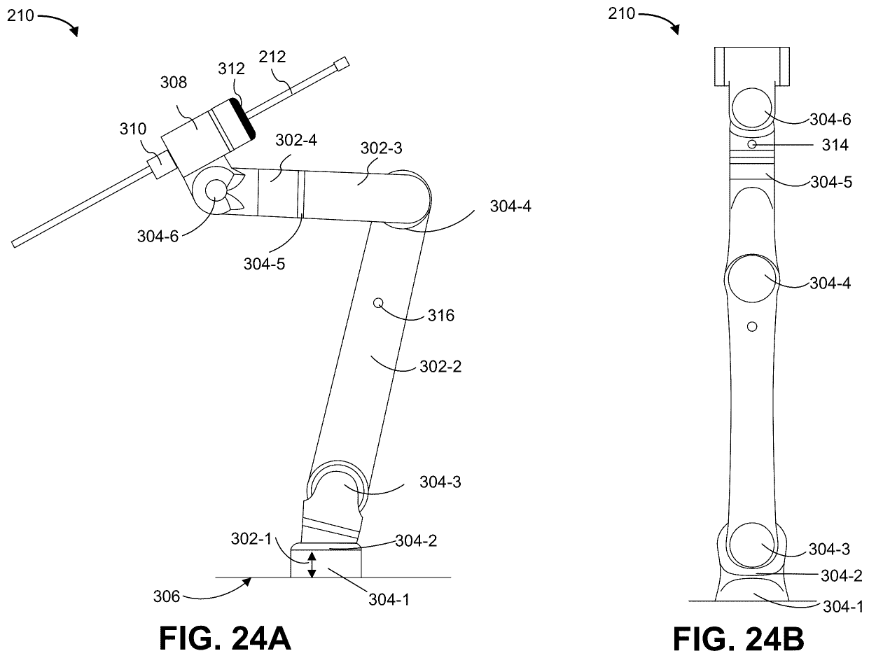

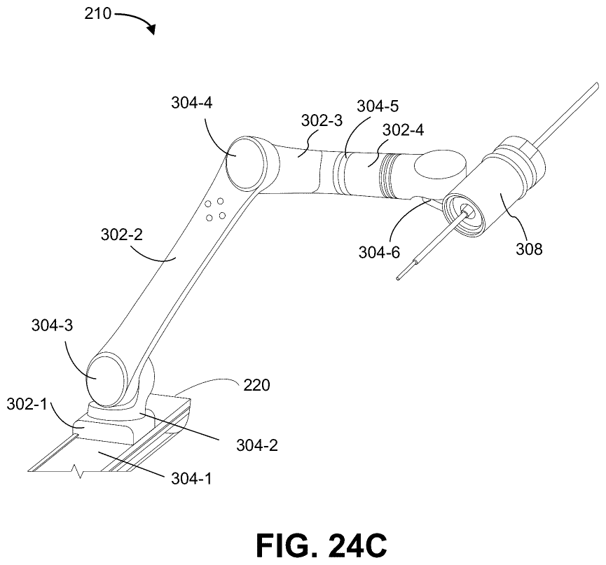

FIGS. 24A to 24C illustrate different views of an exemplary robotic arm 210 according to some embodiments.

FIG. 24A illustrates that the robotic arm 210 includes a plurality of links 302 (e.g., linkages). The links 302 (e.g., 302-1 through 302-4) are connected by one or more joints 304 (e.g., 304-1 through 304-5). Each of the joints 304 includes one or more degrees of freedom (DoFs).

In FIG. 24A, the joints 304 include a first joint 304-1 (e.g., a base joint or an AG joint) that is located at or near a base 306 of the robotic arm 210. In some embodiments, the base joint 304-1 comprises a prismatic joint that allows the robotic arm 210 to translate along the bar 220 (e.g., along the y-axis). The joints 304 also include a second joint 304-2. In some embodiments, the second joint 304-2 rotates with respect to the base joint 304-1. The joints 304 also include a third joint 304-3 that is connected to one end of link 302-2. In some embodiments, the joint 304-3 includes multiple DoFs and facilitates both tilt and rotation of the link 302-2 tilt with respect to the joint 304-3.

FIG. 24A also shows a fourth joint 304-4 that is connected to the other end of the link 302-2. In some embodiments, the joint 304-4 comprises an elbow joint that connects the link 302-2 and the link 302-3. The joints 304 further comprise a pair of joints 304-5 (e.g., a wrist roll joint) and 304-6 (e.g., a wrist pitch joint), which is located on a distal portion of the robotic arm 210.

A proximal end of the robotic arm 210 may be connected to a base 306 and a distal end of the robotic arm 210 may be connected to an advanced device manipulator (ADM) 308 (e.g., a tool driver, an instrument driver, or a robotic end effector, etc.). The ADM 308 may be configured to control the positioning and manipulation of a medical instrument s (e.g., a tool, a scope, etc.).

The robotic arm 210 can also include a cannula sensor 310 for detecting presence or proximity of a cannula to the robotic arm 210. In some embodiments, the robotic arm 210 is placed in a docked state (e.g., docked position) when the cannula sensor 310 detects presence of a cannula (e.g., via one or more processors of the robotic medical system 200). In some embodiments, when the robotic arm 210 is in a docked position, the robotic arm 210 can execute null space motion to maintain a position and/or orientation of the cannula, as discussed in further detail below. Conversely, when no cannula is detected by the cannula sensor 310, the robotic arm 210 is placed in an undocked state (e.g., undocked position).

In some embodiments, and as illustrated in FIG. 24A, the robotic arm 210 includes an input or button 312 (e.g., a donut-shaped button, or other types of controls, etc.) that can be used to place the robotic arm 210 in an admittance mode (e.g., by depressing the button 312). The admittance mode is also referred to as an admittance scheme or admittance control. In the admittance mode, the robotic system 210 measures forces and/or torques (e.g., imparted on the robotic arm 210) and outputs corresponding velocities and/or positions. In some embodiments, the robotic arm 210 can be manually manipulated by a user (e.g., during a set-up procedure, or in between procedures, etc.) in the admittance mode. In some instances, by using admittance control, the operator need not overcome all of the inertia in the robotic medical system 200 to move the robotic arm 210. For example, under admittance control, when the operator imparts a force on the arm, the robotic medical system 200 can measure the force and assist the operator in moving the robotic arm 210 by driving one or more motors associated with the robotic arm 210, thereby resulting in desired velocities and/or positions of the robotic arm 210.

In some embodiments, the links 302 may be detachably coupled to the medical tool 212 (e.g., to facilitate ease of mounting and dismounting of the medical tool 212 from the robotic arm 210). The joints 304 provide the robotic arm 210 with a plurality of degrees of freedom (DoFs) that facilitate control of the medical tool 212 via the ADM 308. In an embodiment as shown in FIG. 22 including multiple robotic arms, each robotic arm can hold its own respective medical tool and pivot the medical tool about a remote center of motion.

FIG. 24B illustrates a front view of the robotic arm 210. FIG. 24C illustrates a perspective view of the robotic arm 210. In some embodiments, the robotic arm 210 includes a second input or button 314 (e.g., a push button) that is distinct from the button 312 in FIG. 24A, for placing the robotic arm 210 in an impedance mode (e.g., by a single press or continuous press and hold of the button 314). In this example, the button 314 is located between the joint 304-5 and the joint 304-6. The impedance mode is also referred to as impedance scheme or impedance control. In the impedance mode, the robotic medical system 200 measures displacements (e.g., changes in position and velocity) and outputs forces and/or torques to facilitate manual movement of the robotic arm. In some embodiments, the robotic arm 210 can be manually manipulated by a user (e.g., during a set-up procedure) in the impedance mode. In some embodiments, under the impedance mode, the operator's movement of one part of a robotic arm 210 may cause motion in one or more joints and/or links throughout the robotic arm 210.

In some embodiments, for admittance control, a force sensor or load cell can measure the force that the operator is applying to the robotic arm 210 and move the robotic arm 210 in a way that feels light. Admittance control may feel lighter than impedance control because, under admittance control, one can hide the perceived inertia of the robotic arm 210 because motors in the controller can help to accelerate the mass. In contrast, with impedance control, the user is responsible for most if not all mass acceleration, in accordance with some embodiments.

In some circumstances, depending on the position of the robotic arm 210 relative to the operator, it may be inconvenient to reach the button 312 and/or the button 314 to activate a manual manipulating mode (e.g., the admittance mode and/or the impedance mode). Accordingly, under these circumstances, it may be convenient for the operator to trigger the manual manipulation mode other than by buttons.

In some embodiments, the robotic arm 210 includes a single button (e.g., the button 312 or 314) that can be used to place the robotic arm 210 in the admittance mode and/or the impedance mode (e.g., by using different presses, such as a long press, a short press, press and hold etc.). In some embodiments, the robotic arm 210 can be placed in impedance mode by a user pushing on arm linkages (e.g., the links 302) and/or joints (e.g., the joints 304) and overcoming a force threshold. In some embodiments, the admittance mode and the impedance mode are common in that they both allow the user to grab the robotic arm 210 and command motion by directly interfacing with it.

In some embodiments, the robotic arm 210 includes an input control for activating an arm follow mode. For example, in some embodiments, the robotic arm 210 can include a designate touch point that is located on a link 302 or a joint 304 of the robotic arm (e.g., an outer shell of the link 302 or a button 316). User interaction (e.g., user touch, contact, etc.) with the designate touch point activates the arm follow mode. In some embodiments, the robotic arm 210 includes multiple touch points. User interaction with any (e.g., one or more) of the touch points activates the arm follow mode.

During a medical procedure, it can be desirable to have the ADM 308 of the robotic arm 210 and/or a remote center of motion (RCM) of the tool 212 coupled thereto kept in a static pose (e.g., position and/or orientation). An RCM may refer to a point in space where a cannula or other access port through which a medical tool 212 is inserted is constrained in motion. In some embodiments, the medical tool 212 includes an end effector that is inserted through an incision or natural orifice of a patient while maintaining the RCM. In some embodiments, the medical tool 212 includes an end effector that is in a retracted state during a setup process of the robotic medical system.

In some circumstances, the robotic medical system 200 can be configured to move one or more links 302 of the robotic arm 210 within a “null space” to avoid collisions with nearby objects (e.g., other robotic arms), while the ADM 308 of the robotic arm 210 and/or the RCM are maintained in their respective poses (e.g., positions and/or orientations). The null space can be viewed as the set of joint states through which a robotic arm 210 can move that does not result in movement of the ADM 308 and/or RCM, thereby maintaining the position and/or the orientation of the medical tool 212 (e.g., within a patient). In some embodiments, a robotic arm 210 can have multiple positions and/or configurations available for each pose of the ADM 308.

For a robotic arm 210 to move an instrument to a desired pose in space, in certain embodiments, the robotic arm 210 may have at least six DoFs—three DoFs for translation (e.g., X, Y, and Z positions) and three DoFs for rotation (e.g., yaw, pitch, and roll). In some embodiments, each joint 304 may provide the robotic arm 210 with a single DoF, and thus, the robotic arm 210 may have at least six joints to achieve freedom of motion to position the ADM 308 at any pose in space. To further maintain the ADM 308 of the robotic arm 210 and/or the remote center or motion in a desired pose, the robotic arm 210 may further have at least one additional “redundant joint.” Thus, in certain embodiments, the system may include a robotic arm 210 having at least seven joints 304, providing the robotic arm 210 with at least seven DoFs. In some embodiments, the robotic arm 210 may include a subset of joints 304 each having more than one degree of freedom thereby achieving the additional DoFs for null space motion. However, depending on the embodiment, the robotic arm 210 may have a greater or fewer number of DoFs.

Furthermore, as described with respect to FIG. 12, the bar 220 (e.g., adjustable arm support) can provide several degrees of freedom, including lift, lateral translation, tilt, etc. Thus, depending on the embodiment, a robotic medical system can have many more robotically controlled degrees of freedom beyond just those in the robotic arms 210 to provide for null space movement and collision avoidance. In a respective embodiment of these embodiments, the end effectors of one or more robotic arms (and any tools or instruments coupled thereto) and a remote center along the axis of the tool can advantageously maintain in pose and/or position within a patient.

A robotic arm 210 having at least one redundant DoF has at least one more DoF than the minimum number of DoFs for performing a given task. For example, a robotic arm 210 can have at least seven DoFs, where one of the joints 304 of the robotic arm 210 can be considered a redundant joint, in accordance with some embodiments. The one or more redundant joints can allow the robotic arm 210 to move in a null space to both maintain the pose of the ADM 308 and a position of an RCM and avoid collision(s) with other robotic arms or objects.

In some embodiments, the robotic medical system 200 can be configured to perform collision avoidance to avoid collision(s), e.g., between adjacent robotic arms 210, by taking advantage of the movement of one or more redundant joints in a null space. For example, when a robotic arm 210 collides with or approaches (e.g., within a defined distance of) another robotic arm 210, one or more processors of the robotic medical system 200 can be configured to detect the collision or impending collision (e.g., via kinematics). Accordingly, the robotic medical system 200 can control one or both of the robotic arms 210 to adjust their respective joints within the null space to avoid the collision or impending collision. In an embodiment including at least a pair of robotic arms, a base of one of the robotic arms and its end effector can stay in its pose, while links or joints therebetween move in a null space to avoid collisions with an adjacent robotic arm.

### C. Exemplary Surgical Settings.

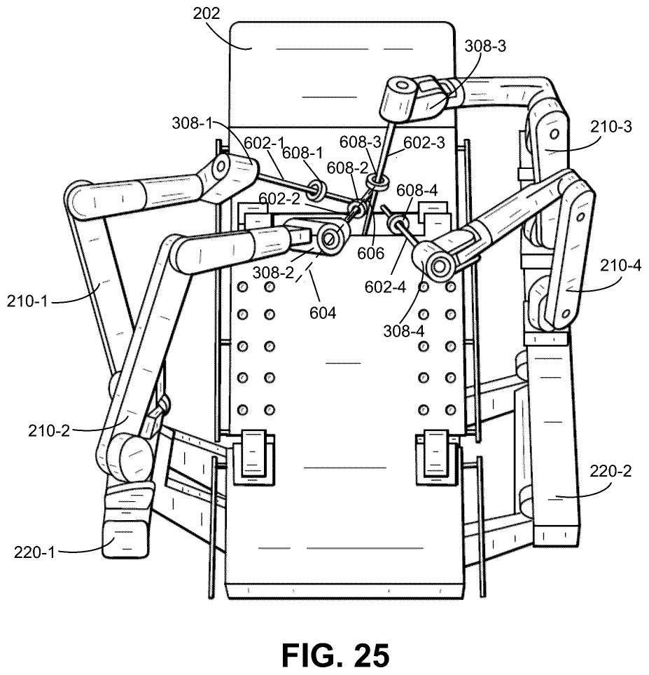

FIG. 25 illustrates a perspective view of a robotic medical system 200 that includes four robotic arms 210-1, 210-2, 210-3, and 210-4, in accordance with some embodiments. Each of the robotic arms 210-1, 210-2, 210-3, and 210-4 is coupled to a respective surgical tool 602 (e.g., 602-1 through 602-4, which may correspond to instrument 212) via a respective ADM 308 (e.g., tool driver), such as ADMs 308-1 through 308-4. A surgical tool 602 can be inserted into a patient via a respective port 608 located on the patient. As used herein, a port (e.g., a port location, a port of entry, an entry point, a port region, a port area, or a port position, etc.) refers to a position on a patient's body through which a medical tool/instrument (e.g., held by a robotic arm) can be inserted and constrained in motion. In some embodiments, the port corresponds to an incision point (or an incision region) that is made through the skin of the patient to facilitate a medical operation or procedure. In some embodiments, the port corresponds to a natural orifice, such as a mouth of the patient (e.g., for a bronchoscopy procedure). In some embodiments, the port corresponds to a medical device with an opening, placed at the incision point or the natural orifice to allow access to a surgical space through the opening. The view of the patient has been excluded from FIG. 25 in order to enhance the visibility of the robotic arms 210 and the surgical tools 602.

In FIG. 25, the robotic arm 210-2 is coupled to a camera 606. In this example, the camera 606 is coupled to the robotic arm 210—via a medical instrument 602-2 (e.g., an endoscope) (e.g., at a distal end of the medical instrument 602-2). In some embodiments, the camera 606 is a part of the medical instrument 602-2. In some embodiments, the camera 606 can be a standalone device (e.g., not part of a surgical instrument) that is coupled to a robotic arm (e.g., the camera 606 is distinct and separate from a medical instrument). In some embodiments, the camera 606 defines an axis 604 (e.g., an optical axis), which identifies an orientation of the camera 606. The camera 606 (or a scope) provides an image of a surgical site to facilitate control of surgical tools to perform a robotic medical procedure. For example, a robotically controllable endoscope of the robotic system can include a camera positioned at a distal tip thereof. The user can view an image from the camera of the endoscope in a viewer in order to facilitate control of the endoscope and/or other components of the robotic medical system. As another example, the robotic system may include one or more cameras laparoscopically or endoscopically inserted into a patient. The user can view images from the inserted cameras in order to facilitate control of one or more additional robotically controlled medical instruments, such as one or more additional laparoscopically inserted medical instruments.

In some embodiments, the robotic medical system 200 includes a coordinate system (e.g., a robot coordinate system, a coordinate frame, a system frame, etc. that may be a Cartesian or non-Cartesian coordinate system), and respective positions of the patient support platform 202, the robotic arms 210, the adjustable arm supports 220, and/or instruments 212 are represented as coordinates (e.g., x-, y-, and z-coordinates) on the coordinate system. For example, the robotic medical system 200 (e.g., one or more processors 380 of the robotic medical system 200) may be configured to identify positions and orientations of the patient support platform 202, the robotic arms 210, the adjustable arm supports 220, and/or instruments 212 based on coordinates in the coordinate system.

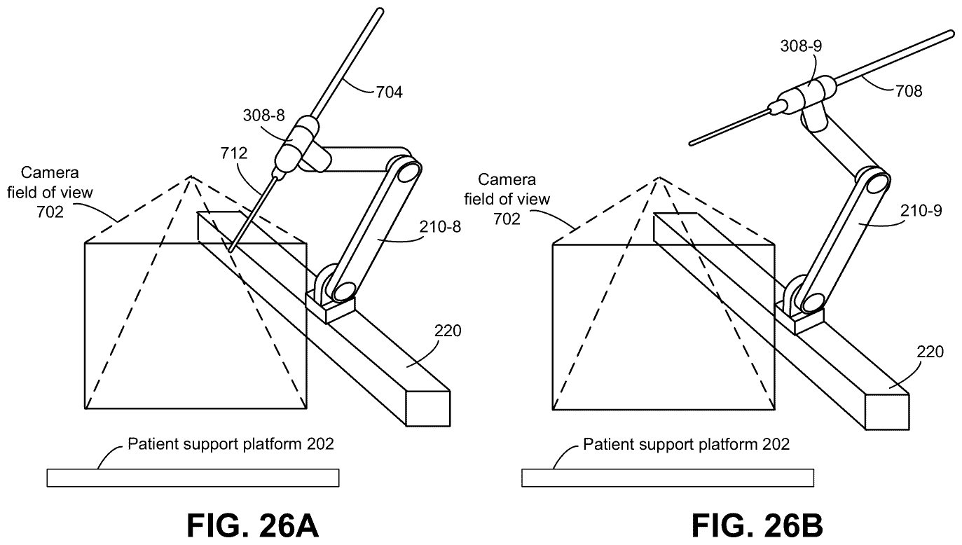

FIGS. 26A and 26B illustrate a camera field of view in a three-dimensional space in accordance with some embodiments. In some embodiments, the robotic medical system 200 determines the field of view based on a focal length of the camera 606 (e.g., the field of view may correspond to a space within a certain distance from a center of the field of view of the camera 606). In some embodiments, the robotic medical system 200 determines the field of view using data obtained by one or more sensors 388 of the robotic medical system 200, such as position (and orientation) data corresponding to the tip of an endoscope (e.g., a medical instrument) to which the camera 606 is coupled or position data corresponding to the camera 606 (e.g., the safety region may be determined to be located at a particular distance from the camera 606). In some embodiments, the robotic medical system 200 stores (e.g., within memory) information identifying the field of view of the camera 606 and utilizes the stored information to determine the field of view of the camera 606 based on the position and orientation of the camera 606. For example, the robotic medical system 200 may store information identifying a predefined height (e.g., several centimeters) and predefined base angles, slant angles, and/or edge angles of a three-dimensional volume, corresponding to the field of view, having a shape of a truncated rectangular pyramid. In another example, the robotic medical system 200 may store information identifying a predefined radius or diameter and a predefined distance (e.g., several centimeters) for a spherical volume, corresponding to the field of view, located at the predefined distance from the camera 606 and having the predefined radius or diameter. In some embodiments, the robotic medical system 200 determines the field of view according to image(s) obtained using the camera 606.

In FIGS. 26A and 26B, the camera field of view 702 (e.g., as seen by a camera that is coupled to another robotic arm) is represented by a three-dimensional space (e.g., a pyramid). It would be appreciated by one of ordinary skill in the art that the three-dimensional space of a camera field of view can be represented by other shapes such as a cone, a cylinder, a sphere, a tetrahedron, etc. In the example of FIG. 26A, the robotic arm 210-8 is coupled to a surgical tool 704 (e.g., a medical instrument) via ADM 308-8. The surgical tool 704 intersects (e.g., overlaps, is within, etc.) the three-dimensional space of the camera field of view 702. In contrast, FIG. 26B shows a robotic arm 210-9 holding a surgical tool 708 (e.g., via ADM 308-9) that does not intersect the three-dimensional space of the camera field of view 702 (e.g., because of the position and/or orientation of the tool 708).

### D. Exemplary User Interfaces.

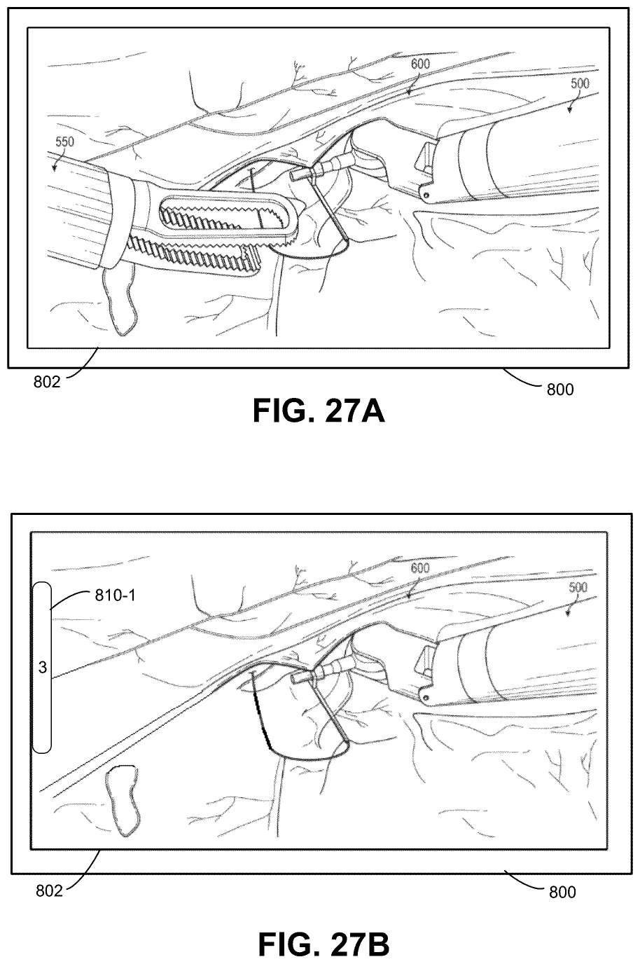

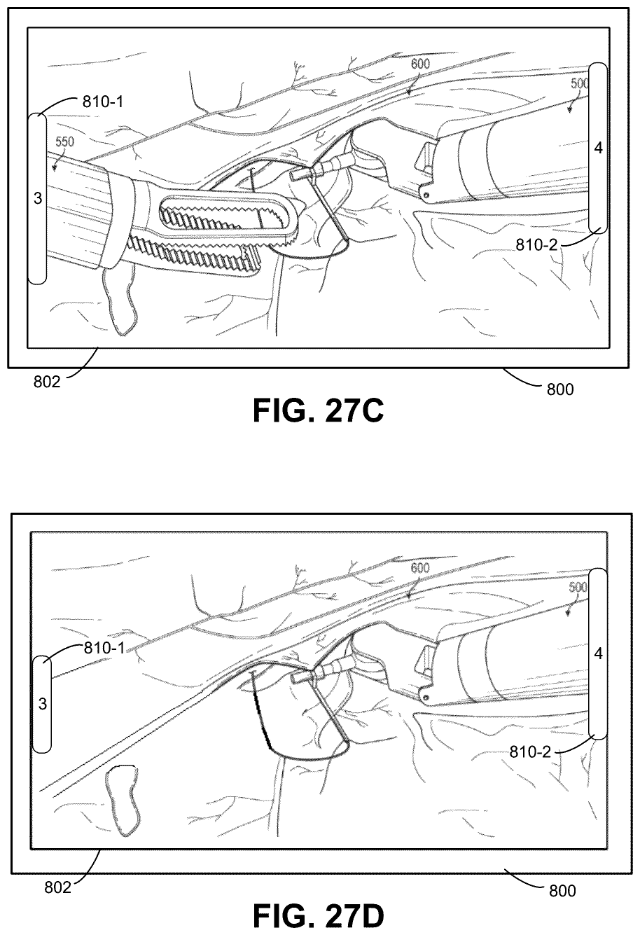

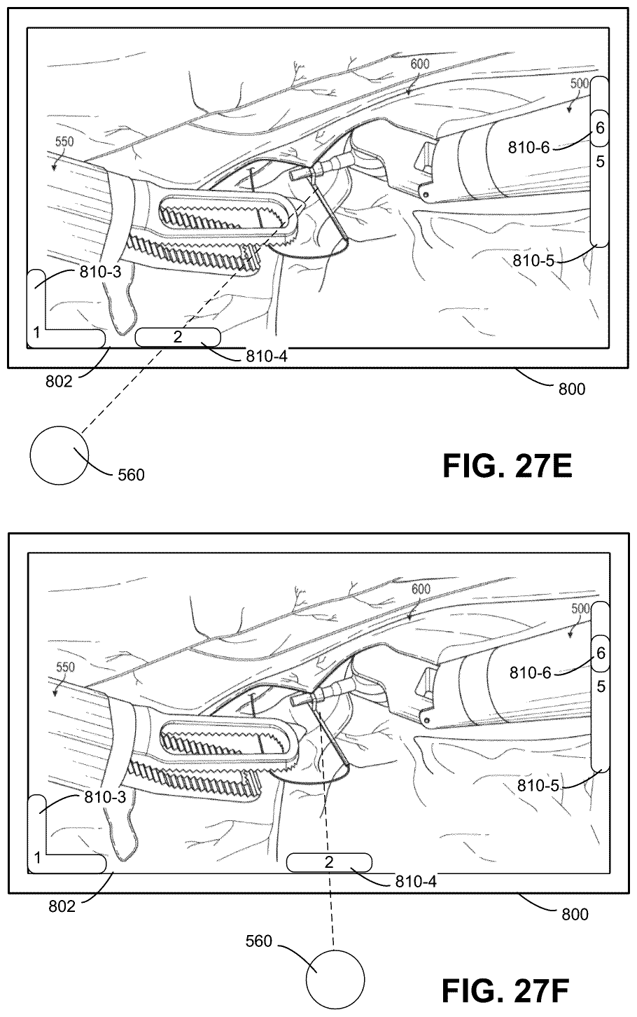

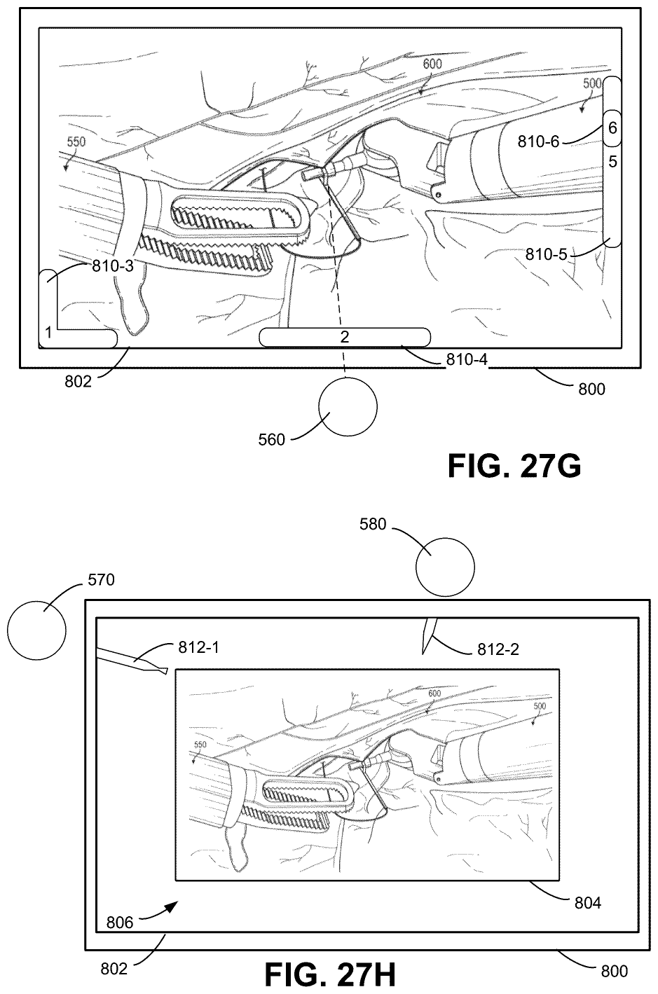

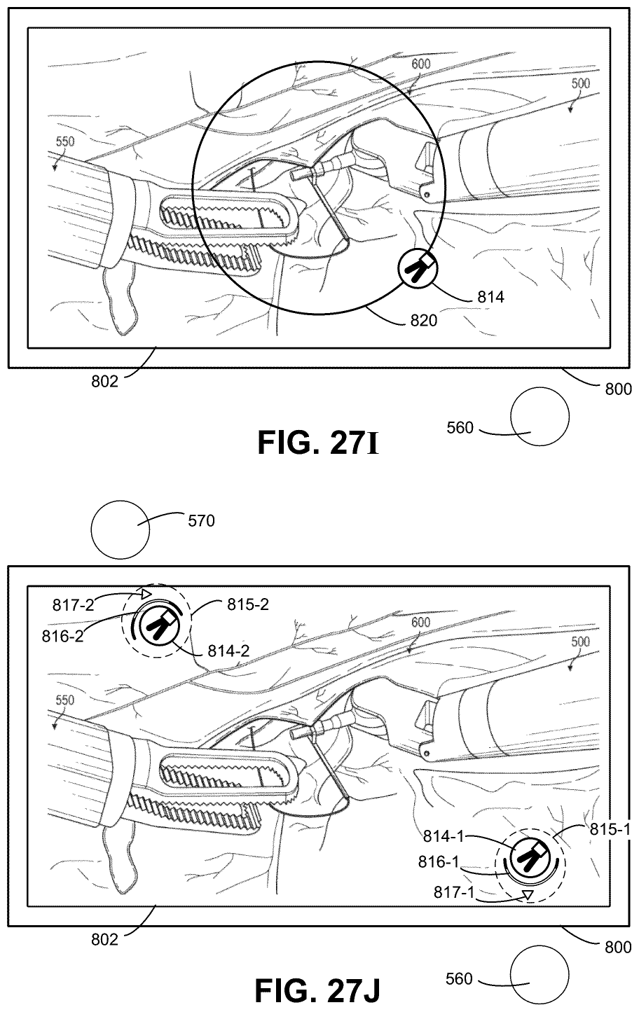

FIGS. 27A to 27J illustrate exemplary user interfaces displayed on a display device in accordance with some embodiments.

FIG. 27A shows a user interface 802 displayed on a display device 800 in accordance with some embodiments. In some embodiments, as shown in FIG. 27A, the user interface 802 includes an image or representation of a field of view of a camera (e.g., a view of a surgical site, such as a treatment site 600) or a scope. FIG. 27A also shows that the user interface 802 includes images or representations of medical instruments 500 and 550 located within the field of view of the camera or scope.

FIG. 27B shows that the user interface 802 is updated in response to the medical instrument 550 moving out of the field of view. As a result, the user interface 802 in FIG. 27B does not include an image or presentation of the medical instrument 550. In addition, FIG. 27B shows that the user interface 802 is updated (e.g., in response to the medical instrument 550 moving out of the field of view) to include an indicator 810-1 (e.g., along a periphery of the user interface 802 or a portion thereof) for indicating a position (or direction) of the medical instrument 550 relative to the field of view. For example, in FIG. 27B, the indicator 810-1 is located along a left edge of the user interface 802, indicating that the medical instrument 550 is positioned on a left side of the field of view. In some embodiments, as shown in FIG. 27B, the indicator 810-1 includes information identifying the medical instrument 550 (e.g., a number associated with a robotic arm coupled with the medical instrument 550, such as the number “3” indicating a third robotic arm). While the indicator 810-1 is illustrated as being near or adjacent a rectangular border, in some embodiments, the indicator 810-1 can be placed further inward away from the rectangular border.

In some embodiments, when the medical instrument 550 moves back into the field of view, the user interface 802 is updated to include an image or representation of the medical instrument 550. In some embodiments, the user interface 802, in response to the medical instrument 550 moving back into the field of view, ceases to include the indicator 810-1 (e.g., the indicator 810-1 ceases to be displayed in response to the medical instrument 550 moving back into the field of view). Thus, in some embodiments, the indicator 810-1 (or any other indicator for any medical tool) is displayed only when the corresponding medical tool is located outside the field of view.

In some embodiments, the indicator 810-1 (or any other indicator for any medical tool) is displayed regardless of whether the corresponding medical tool is located within or outside the field of view. For example, FIG. 27C shows that the user interface 802 includes the indicator 810-1 associated with the medical tool 550 and the indicator 810-2 associated with the medical tool 500 even when the user interface 802 includes images or representations of the medical tools 500 and 550.

FIG. 27D shows that the indicator 810-1 is updated to indicate a distance between the medical tool 550 and the field of view. For example, in some embodiments, in response to the medical tool 550 moving out of the field of view, the size of the indicator 810-1 is reduced (e.g., the indicator 810-1 becomes shorter and/or narrower) to indicate an increased distance between the medical tool 550 and the field of view.

Similarly, the user interface 802 shown in FIG. 27B when the medical instrument 550 is outside the field of view may be updated as shown in FIG. 27D to decrease the size of the indicator 810-1, indicating that the distance between the medical instrument 550 and the field of view has increased. Accordingly, the indicator 810-1 can represent a dynamic indicator that can either increase or decrease in size as a surgical procedure progresses.

FIG. 27E shows that the user interface 802 includes images or representations of medical tools 550 and 550 located within the field of view. The user interface 802 also includes indicators 810-3 through 810-6 corresponding to medical tools located outside the field of view. For example, the indicator 810-4 indicates a position (or direction) of a medical tool 560 relative to the field of view.

FIG. 27F shows that the user interface 802 is updated in response to a (e.g., lateral) movement of the medical tool 560 relative to the field of view. For example, the indicator 810-4 moves within the user interface 802 (e.g., along a periphery of the user interface 802) to indicate an updated position (or direction) of the medical tool 560 relative to the field of view.

FIG. 27G shows that the user interface 802 is updated in response to a movement of the medical tool 560 relative to the field of view, changing a distance between the medical tool 560 and the field of view. In particular, the shape of the indicator 810-4 may be updated to indicate a distance between the medical tool 560 (located outside the field of view) and the field of view. For example, in FIG. 27G, the size of the indicator 810-4 has increased (e.g., the indicator 810-4 becomes longer and/or wider) to indicate that the distance between the medical tool 560 and the field of view has decreased. Alternatively, the size of the indicator 810-4 may decrease (e.g., the indicator 810-4 becomes shorter and/or narrower) to indicate that the distance between the medical tool 560 and the field of view has increased. Additionally or alternatively, a text associated with the indicator 810-4 may be displayed to indicate a distance between the medical tool 560 and the field of view (e.g., text, such as “close,” “medium,” or “far,” may be displayed based on a comparison of a distance between the medical tool 560 and the field of view and one or more distance thresholds). In some embodiments, a color of the indicator 810-4 may change to indicate a distance between the medical tool 560 and the field of view (e.g., a first color, such as green, to indicate that a distance between the medical tool 560 and the field of view is below a distance threshold and a second color, such as yellow, to indicate that the distance between the medical tool 560 and the field of view is above the distance threshold).

FIG. 27H shows the user interface 802 including an image or representation of the field of view along with images or representations of medical tools located outside the field of view. In the user interface 802 shown in FIG. 27H, the image or representation of the field of view is surrounded by a margin 806 in which representations 812-1 and 812-2 of medical tools 570 and 580 located outside the field of view are shown. In some embodiments, the images or representations of the medical tools located outside the field of view identify the types and/or orientations of the medical tools. For example, the images or representations of the medical tools may include distinct images or representations for respective types of medical tools (e.g., the representation 812-1 may identify a medical tool of a first type, such as a clamp, and the representation 812-2 may identify a medical tool of a second type that is distinct from the first type, such as a suturing device).

FIG. 27I shows the user interface 802 including a user interface element highlighting a portion of the user interface 802 (e.g., a focal point). For example, the user interface 802 in FIG. 27I includes the user interface element 820 highlighting a central region of the user interface 802. In FIG. 27I, the user interface element 820 has a shape of a circle, although the user interface element 820 may have a shape of an ellipse, a rectangle (with or without rounded corners), a square, a triangle, a pentagon, a hexagon, or any other shape. In some embodiments, the user interface element 820 highlights the portion of the user interface 802 by indicating a boundary of the portion of the user interface 802 (e.g., a circular boundary). In some embodiments, the user interface element 820 highlights the portion of the user interface 802 by changing the appearance of the portion of the user interface 802 or the rest of the user interface 802 other than the portion of the user interface 802 (e.g., by changing the brightness, color contrast, or color tone).

The user interface 802 in FIG. 27I also includes an indicator 814 indicating a position (or direction) of the medical tool 560 relative to the field of view. In some embodiments, the indicator 814 is positioned adjacent to a periphery of the user interface element 820. In some embodiments, the indicator 814 may move along the periphery of the user interface element 820 in response to a movement of the medical tool 560 changing the direction of the medical tool 560 from the field of view. In some embodiments, the indicator 814 may be updated (e.g., the size, color, or shape of the indicator 814 may change) to indicate a change in the distance between the medical tool 560 and a portion of the field of view corresponding to the highlighted portion of the user interface 802.

FIG. 27J also shows the user interface 802 including an image or representation of the field of view along with indicators 815-1 and 815-2. In some embodiments, the indicators 815-1 and 815-2 include images or representations 814-1 and 814-2 of medical tools 560 and 570 located outside the field of view. In some embodiments, the indicators 815-1 and 815-2 include additional user interface elements for indicating the direction and/or distance to corresponding medical tools. For example, in FIG. 27J, the indicator 815-1 includes a user interface element 816-1 indicating a distance between the field of view and the medical tool 560. The user interface element 816-1 may be shown adjacent to the representation 814-1 of the medical tool 560. In some embodiments, the user interface element 816-1 includes a line (e.g., a straight line or a curved lien, such as an arc), the length of which indicates the distance between the field of view and the medical tool 560. In some embodiments, the orientation of the user interface element 816-1 also indicates the direction of the medical tool 560 relative to the field of view. In FIG. 27J, the indicator 815-1 also includes a user interface element 817-1 indicating the direction of the medical tool 560 relative to the field of view. The user interface element 817-1 may be shown adjacent to the representation 814 of the medical tool 560. In some embodiments, the user interface element 817-1 has a shape suitable for indicating a direction (e.g., the user interface element 817-1 may have a shape of an arrow, a triangle, etc.). In some embodiments, the indicator 815-1 includes the user interface element 816-1 without the user interface element 817-1. In some embodiments, the indicator 815-1 includes the user interface element 817-1 without the user interface element 816-1. In some embodiments, the indicator 815-1 includes both the user interface element 816-1 and the user interface element 817-1, as shown in FIG. 27J. Similarly, the indicator 815-2 shown in FIG. 27J also includes both a user interface element 816-2 indicating a distance between the field of view and the medical tool 570 and a user interface element 817-2 indicating the direction of the medical tool 570 relative to the field of view, shown adjacent to the representation 814 of the medical tool 570. The use of a separate user interface element (e.g., the user interface element 816-1) for indicating the distance between the field of view and a corresponding medical tool may facilitate the user to ascertain the distance between the field of view and the corresponding medical tool easily. The use of a separate user interface element (e.g., the user interface element 817-1) for indicating the direction from the field of view to a corresponding medical tool allows placement of the image or representation (e.g., the representation 814) of the corresponding medical tool at locations within the user interface 802 independent of the direction from the field of view to the corresponding medical tool, which, in turn, allows placement of the indicator away from a center region of the field of view.

Although certain aspects of indicators may have been described with respect to a particular example of an indicator (e.g., indicator 810-4), such aspects may be applicable to any other indicators described with respect to FIGS. 27A through 27J.

Although FIGS. 27E through 27J illustrate one or more medical tools (e.g., medical tools 560, 570, and 580), located outside the field of view, relative to the user interface 802 shown on the display device 800, a person having ordinary skill in the art would understand that positions (or directions) of one or more medical tools shown relative to the user interface 802 in FIGS. 27E through 27J represent positions (or directions) of the one or more medical tools relative to the field of view at the surgical site.

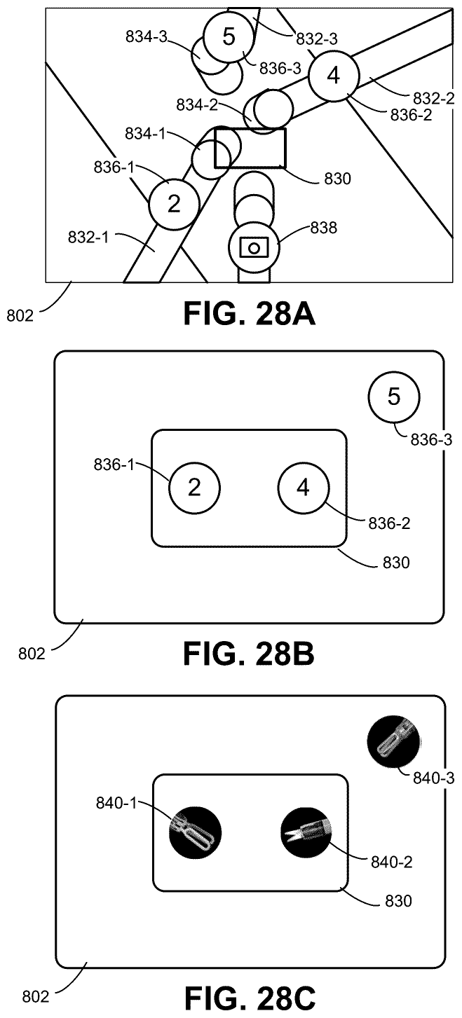

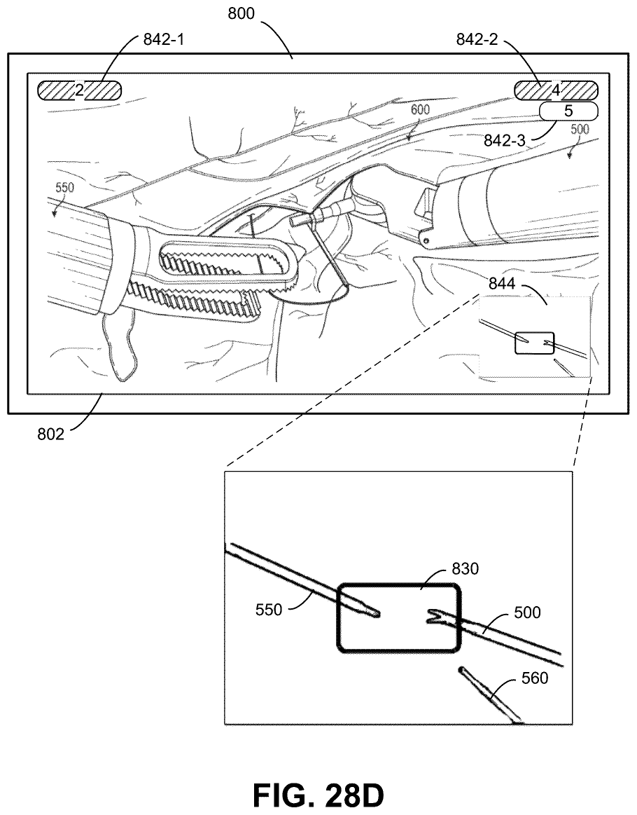

FIGS. 28A to 28D illustrate exemplary user interfaces displayed on a display device in accordance with some embodiments.

FIG. 28A shows a birds-eye view of a robotic medical system shown in the user interface 802 in accordance with some embodiments. The birds-eye view shown in FIG. 28A includes robotic arms 832-1, 832-2, and 832-3. The birds-eye view shown in FIG. 28A also includes an ADM (e.g., 834-1, 834-2, and 834-3) coupled with each robotic arm 832. In some embodiments, each ADM indicates a position of a medical tool coupled thereto. In some embodiments, the birds-eye view also includes information identifying each robotic arm (e.g., robotic arm numbers in indicators 836-1, 836-2, and 836-3). In some embodiments, the birds-eye view includes an image or representation of a camera or scope 838 for indicating a position and/or orientation of the camera or scope 838. The birds-eye view can facilitate determining potential collisions between the robotic arms, ADMs, and/or associated tools. In some embodiments, as shown in FIG. 28A, the birds-eye view may include a visual indication 830 of the field of view of the camera or scope. In some embodiments, the birds-eye view highlights which of the tools is presently under the control of the surgeon (or “active”).

FIG. 28B shows a graphical representation of the birds-eye view in accordance with some embodiments. In some embodiments, the user interface 802 includes the visual indication 830 of the field of view and one or more indicators for corresponding medical tools. For example, in FIG. 28B, the user interface 802 includes indicators 836-1 and 836-2, for medical tools located within the field of view (e.g., medical tools held by ADMs 834-1 and 834-2), inside the visual indication 830 and indicator 836-3, for a medical tool located outside the field of view (e.g., medical tool held by ADM 834-3), outside the visual indication 830. The indicator 836-3 is located to indicate the position of the corresponding medical tool relative to the field of view (e.g., the medical tool held by ADM 834-3 is positioned in the upper-right direction from the field of view). In some embodiments, the birds-eye view highlights which of the tools is presently under the control of the surgeon (or “active”).

In FIG. 28B, the indicators 836-1, 836-2, and 836-3 include information identifying associated robotic arms (e.g., robotic arm numbers). FIG. 28C is similar to FIG. 28B, except that, in FIG. 28C, the user interface 802 includes indicators 840-1, 840-2, and 840-3 graphically identifying types of associated medical tools instead of indicators 836-1, 836-2, and 836-3. For example, indicator 840-1 may identify a grasper, indicator 840-2 may identify scissors, and indicator 840-3 may identify a grasper. In some embodiments, the birds-eye view highlights which of the tools is presently under the control of the surgeon (or “active”).

In some embodiments, the user interface 802 may include indicators that both identify types of associated medical tools and associated robotic arms (e.g., a combination of a graphical representation of a type of associated medical tool and a robotic arm number).

FIG. 28D shows that the user interface 802 includes a concurrent display of (i) an image or representation of the field of view with (ii) a graphical representation 844 of medical tools located within and outside the field of view. In FIG. 28D, the graphical representation 844 includes (a) the visual indication 830 of the field of view and (b) respective graphical representations of medical tools 500, 550, and 560. In some embodiments, the graphical representations of medical tools 500, 550, and 560 identify the directions, orientations, and/or types of medical tools 500, 550, and 560.

In some embodiments, instead of the graphical representation 844, any of the graphical representations shown in FIGS. 28A, 28B, and 28C may be displayed concurrently with the image or representation of the field of view (e.g., the graphical representation shown in FIG. 28A, 28B, or 28C may be shown as an inset in place of the graphical representation 844 shown in FIG. 28D). In some embodiments, the graphical representation 844 or any of the graphical representations shown in FIGS. 28A, 28B, and 28C may be displayed side-by-side with the image or representation of the field of view.

In some embodiments, the user interface 802 also includes indicators 842-1, 842-2, and 842-3. In some embodiments, each of the indicators 842-1, 842-2, and 843-3 is shown adjacent to a left-side corner (e.g., a top-left corner or a bottom-left corner) or a right-side corner (e.g., a top-right corner or a bottom-right corner) depending on a position of a corresponding tool relative to a center of the field of view (e.g., the indicator 842-1 is shown in the top-left corner as the corresponding medical tool 550 is positioned on a left side from the center of the field of view and the indicators 842-2 and 842-3 are shown in the top-right corner as the corresponding medical tools 500 and 560 are positioned on a right side from the center of the field of view). In some embodiments, one or more indicators for medical tools located within the field of view are visually distinguishable (e.g., based on the color, size, highlighting, etc.) from one or more indicators for medical tools located outside the field of view (e.g., the indicators 842-1 and 842-2 for medical tools 550 and 500 located within the field of view are highlighted, whereas the indicator 842-3 for medical tool 560 located outside the field of view is not highlighted). In some embodiments, the size, shape, and/or color of an indicator are updated to indicate a distance between the corresponding medical tool and the field of view (e.g., the size of indicator 842-3 is reduced to indicate a distance between the medical tool 560 and the field of view).

In some embodiments, a user (e.g., a surgeon) may switch on or off display of indicators. For example, the user may provide a request for display of indicators or cessation of the display of indicators via one or more input devices (e.g., one or more user interface elements, such as buttons, displayed on a display device, the foot pedal 244, etc.). In some configurations, the user may provide a request for display of indicators or cessation of the display indicators via a voice command. In some embodiments, the user may switch from indicators of a first type to indicators of a second type distinct from the first type (e.g., indicators shown in FIG. 27G to indicators shown in FIG. 27H) via the one or more input devices (or via a voice command).

As described above, FIGS. 27A-27J and 28A-28D illustrate exemplary user interfaces. A person having ordinary skill in the art would understand that one or more processors (e.g., central processing unit, graphics processing unit, accelerated processing unit, application-specific integrated circuit, etc.) may be configured to generate electrical signals (e.g., video signals) so that one or more display devices can display the user interfaces illustrated in FIGS. 27A-27J and 28A-28D.

### E. Exemplary Processes for Displaying Off-Screen Indicators.

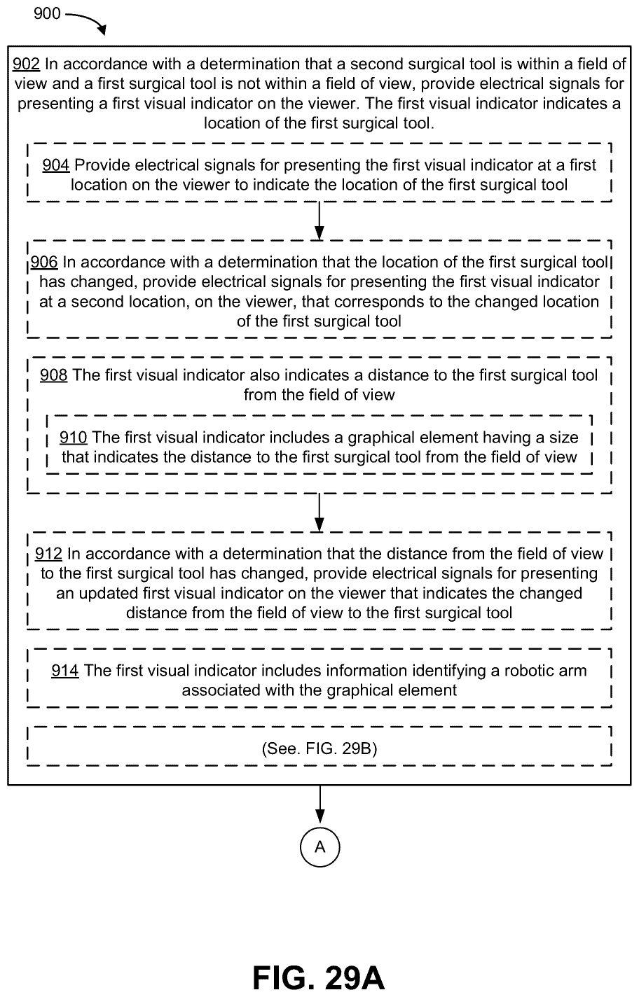

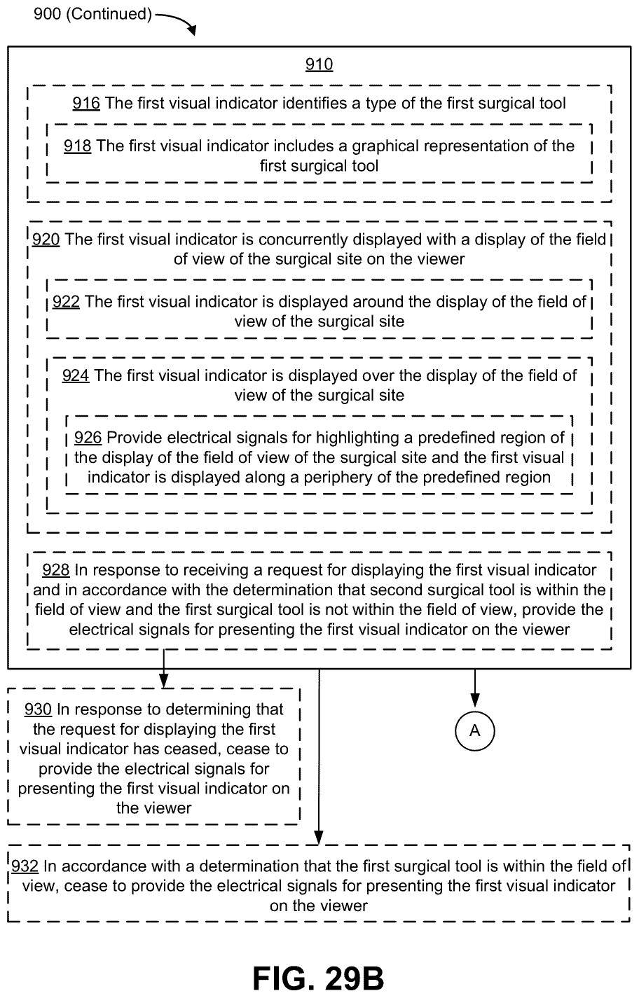

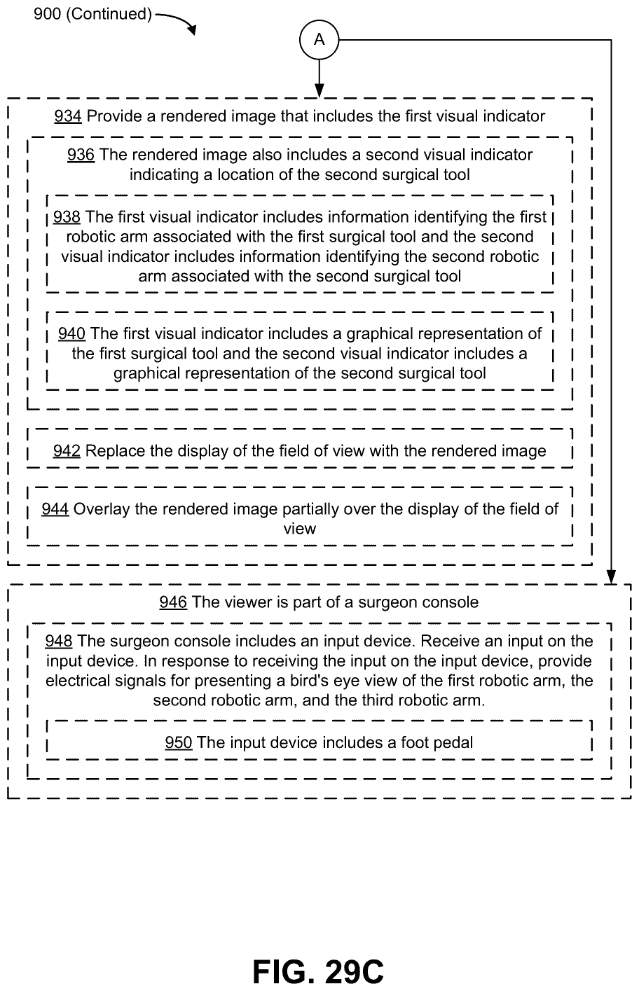

FIGS. 29A to 29C illustrate a flowchart diagram for a method 900 performed by one or more processors (e.g., processors 380) of a surgical robotic system (e.g., the robotic medical system 200 as illustrated in FIGS. 21 and 22, or a robotic surgery platform, etc.), in accordance with some embodiments. The surgical robotic system includes memory that stores instructions for execution by the one or more processors.

The surgical robotic system includes a first robotic arm (e.g., a robotic manipulator) (e.g., the robotic arm 210-1 in FIG. 22) coupled to a first surgical tool (e.g., medical instrument 212 in FIGS. 21 and 24A, surgical tools 602 in FIG. 25, and surgical tool 704 and 708 in FIGS. 26A and 26B).

The surgical robotic system includes a second robotic arm (e.g., a robotic manipulator) (e.g., the robotic arm 210-2 in FIG. 22) coupled to a second surgical tool (e.g., medical instrument 212 in FIGS. 21 and 24A, surgical tools 602 in FIG. 25, and surgical tool 704 and 708 in FIGS. 26A and 26B).

The surgical robotic system includes a third robotic arm (e.g., a robotic manipulator) (e.g., the robotic arm 210-1 in FIG. 22) coupled to a scope (e.g., camera 606).

The robotic system also includes a viewer (e.g., display device 232 located on a tower 230, or display device 242 that is included with a physician console 240) for displaying a field of view of a surgical site derived from the scope.

The robotic system further includes one or more processors (e.g., processor 380) and memory (e.g., memory 382) storing instructions for execution by the one or more processors.

The surgical robotic system, in accordance with a determination that the second surgical tool is within the field of view and the first surgical tool is not within the field of view, (902) provides electrical signals (e.g., video signals, such as analog video signals or digital video signals) for presenting a first visual indicator on the viewer (e.g., the user interface 802 shown in FIG. 27B with the indicator 810-1). The first visual indicator indicates a location (or direction) of the first surgical tool (e.g., the indicator 810-1 indicates the direction of the medical tool 550 relative to the field of view).

In some embodiments, the surgical robotic system (904) provides electrical signals for presenting the first visual indicator at a first location on the viewer to indicate the location (or direction) of the first surgical tool; and, subsequent to providing the electrical signals for presenting the first visual indicator at the first location on the viewer, in accordance with a determination that the location (or direction) of the first surgical tool has changed, (906) provides electrical signals for presenting the first visual indicator at a second location, on the viewer, that corresponds to the changed location (or direction) of the first surgical tool. For example, as shown in FIGS. 27E and 27F, the surgical robotic system changes the location of the indicator 810-4 to indicate the changed direction of the medical tool 560 relative to the field of view.

In some embodiments, the first visual indicator also (908) indicates a distance to the first surgical tool from the field of view (e.g., the size, color, and/or shape of the first visual indicator and/or associated text may indicate the distance to the first surgical tool from the field of view). In some embodiments, the distance to the first surgical tool from the field of view corresponds to a distance between the first surgical tool and a point on a boundary of the field of view (e.g., a position on the boundary of the field of view that is the closest to the first surgical tool). For example, the distance to the first surgical tool from the field of view corresponds to a shorted distance between the first surgical tool and any point on a boundary of the field of view. In some embodiments, the distance to the first surgical tool from the field of view corresponds to a distance between the first surgical tool and a center of the field of view.

In some embodiments, the surgical robotic system updates the first visual indicator in real time to indicate the location (or direction) of the first surgical tool and/or the distance between the first surgical tool and the field of view while the first surgical tool is moving relative to the field of view.

In some embodiments, the first visual indicator (910) includes a graphical element having a size that indicates the distance to the first surgical tool from the field of view (e.g., FIGS. 27F and 27G).

In some embodiments, the surgical robotic system, subsequent to providing the electrical signals for presenting the first visual indicator on the viewer, in accordance with a determination that the distance from the field of view to the first surgical tool has changed, (912) provides electrical signals for presenting an updated first visual indicator on the viewer that indicates the changed distance from the field of view to the first surgical tool (e.g., the size, color, and/or shape of the first visual indicator and/or associated text may be updated to indicate the changed distance between the first surgical tool and the field of view). For example, as shown in FIGS. 27F and 27G, the surgical robotic system changes the size of the indicator 810-4 to indicate the changed distance between the medical tool 560 and the field of view.

In some embodiments, the first visual indicator (914) includes information identifying a robotic arm associated with the graphical element (e.g., as shown in FIG. 27B, the indicator 810-1 may include a number associated with the robotic arm).

In some embodiments, the first visual indicator (916) identifies a type of the first surgical tool. For example, in some embodiments, the first visual indicator (918) includes a graphical representation of the first surgical tool (e.g., representation 812-1 in FIG. 27H, indicator 814 in FIG. 27I, etc.).

In some embodiments, the first visual indicator is (920) concurrently displayed with a display of the field of view of the surgical site on the viewer (e.g., FIGS. 27B-27J and 28D).

In some embodiments, the first visual indicator is (922) displayed around the display of the field of view of the surgical site (e.g., in FIG. 27H, the indicator 810-1 is displayed around the display 804 of the field of view of the surgical site). In some embodiments, the first visual indicator is displayed around, and separated from, the display of the field of view of the surgical site (e.g., FIG. 27H). In some embodiments, the first visual indicator is displayed around, and in contact with, the display of the field of view of the surgical site (e.g., FIG. 27G).

In some embodiments, the first visual indicator is (924) displayed over the display of the field of view of the surgical site (e.g., FIG. 28D).

In some embodiments, the surgical robotic system (926) provides electrical signals for highlighting a predefined region of the display of the field of view of the surgical site (e.g., as shown in FIG. 27I, the user interface 802 includes the user interface element 820 highlighting a central region of the user interface 802). The first visual indicator is displayed along a periphery of the predefined region (e.g., indicator 814 located along a periphery of the user interface element 820 or the region of the user interface 802 defined by the user interface element 820).

In some embodiments, the surgical robotic system, in response to receiving a request for displaying the first visual indicator and in accordance with the determination that second surgical tool is within the field of view and the first surgical tool is not within the field of view, (928) provides the electrical signals for presenting the first visual indicator on the viewer. For example, the surgical robotic system may display the first visual indicator only in response to receiving a request for displaying the first visual indicator (e.g., a user input on one or more input devices). In some embodiments, the surgical robotic system, in response to receiving the request for displaying the first visual indicator (and, in some embodiments, regardless of the determination that second surgical tool is within the field of view and the first surgical tool is not within the field of view), provides the electrical signals for presenting the first visual indicator on the viewer.

In some embodiments, the surgical robotic system, in response to determining that the request for displaying the first visual indicator has ceased, (930) ceases to provide the electrical signals for presenting the first visual indicator on the viewer. For example, the surgical robotic system may cease to display the first visual indicator in response to determining that the request for displaying the first visual indicator has ceased. For example, the request for displaying the first visual indicator may include pressing a button or a pedal, and the surgical robotic system may display the first visual indicator while the button or the pedal is being pressed, and ceases to display the first visual indicator in accordance with a determination that the button or the pedal has been released.

In some embodiments, the surgical robotic system, while the robotic system is in an indicator display mode, and in accordance with the determination that second surgical tool is within the field of view and the first surgical tool is not within the field of view, provides the electrical signals for presenting the first visual indicator on the viewer; and while the robotic system is not in the indicator display mode, ceases to provide the electrical signals for presenting the first visual indicator on the viewer. For example, the display of the indicators may be turned on or off. Once the display of the indicators is turned on (e.g., the surgical robotic system is in the indicator display mode), the surgical robotic system displays the indicators (e.g., as shown in FIG. 27B). Once the display of the indicators is turned off (e.g., the surgical robotic system is not in the indicator display mode), the surgical robotic system does not display the indicators (regardless of whether one or more tools are located outside the field of view). In some embodiments, the surgical robotic system ceases to determine whether one or more tools are located outside the field of view while the surgical robotic system is not in the indicator display mode.

In some embodiments, the surgical robotic system switches into or out of the indicator display mode in response to a user input. For example, in response to a request for display of indicators via one or more input devices (e.g., one or more user interface elements, such as buttons, displayed on a display device, the foot pedal 244, a voice command, etc.), the surgical robotic system switches to (or activates) the indicator display mode. Similarly, in response to a request for cessation of the display of indicators via one or more input devices (e.g., one or more user interface elements, such as buttons, displayed on a display device, the foot pedal 244, a voice command, etc.), the surgical robotic system switches out of (or deactivates) the indicator display mode.

In some embodiments, the surgical robotic system, subsequent to providing the electrical signals for presenting the first visual indicator on the viewer, in accordance with a determination that the first surgical tool is within the field of view, (932) ceases to provide the electrical signals for presenting the first visual indicator on the viewer (e.g., the surgical robotic system provides electrical signals for presenting a user interface that does not include the first visual indicator). For example, as described with respect to FIGS. 27A and 27B, in some embodiments, in response to the surgical tool 550 returning to the field of view, the indicator 810-1 ceases to be displayed.

In some embodiments, the surgical robotic system (934) provides a rendered image that includes the first visual indicator (e.g., FIG. 28B is a rendered image that includes the indicator 836-3 indicating the location (or direction) of the tool associated with the ADM 834-3).

In some embodiments, the rendered image also (936) includes a second visual indicator indicating a location (or direction) of the second surgical tool (e.g., in FIG. 28B, the rendered image includes the indicator 836-1 indicating the location of the tool associated with the ADM 834-1).

In some embodiments, the first visual indicator (938) includes information identifying the first robotic arm associated with the first surgical tool and the second visual indicator includes information identifying the second robotic arm associated with the second surgical tool (e.g., in FIG. 28B, the indicators 836-3 and 836-1 include associated robotic arm numbers).

In some embodiments, the first visual indicator (940) includes a graphical representation of the first surgical tool and the second visual indicator includes a graphical representation of the second surgical tool (e.g., in FIG. 28C, indicators 840-3 and 840-1 includes graphical representations of associated medical tools).

In some embodiments, the surgical robotic system (942) replaces the display of the field of view with the rendered image. For example, the surgical robotic system may display the field of view (e.g., as part of the user interface 802 shown in any of FIGS. 27A through 27J), and subsequently replace the display of the field of view with the rendered image in response to receiving a request from a user (e.g., a user input on one or more input devices) to display the rendered image.

In some embodiments, the surgical robotic system (944) overlays the rendered image partially over the display of the field of view (e.g., as shown in FIG. 28D, the rendered image is laid over a portion, less than all, of the display of the field of view).

In some embodiments, the viewer is (946) part of a surgeon console (e.g., the display device 242 of the physician console 240).

In some embodiments, the surgeon console includes an input device (e.g., buttons, switches, touch-sensitive surfaces, gimbals, pedals, etc.). The surgical robotic system (948) receives an input on the input device (e.g., pressing on a button or a pedal); and in response to receiving the input on the input device, provides electrical signals for presenting a bird's eye view (e.g., the user interface shown in FIG. 28A) of the first robotic arm, the second robotic arm, and the third robotic arm.

In some embodiments, the input device includes a foot pedal (e.g., the foot pedal 244 shown in FIG. 23).

In some embodiments, the first robotic arm and the second robotic arm are positioned bilaterally relative to an operating table (e.g., the robotic arms 210-1 and 210-4 in FIG. 22).

In some embodiments, the first robotic arm is supported on (e.g., movably coupled to) a first adjustable arm support and the second robotic arm is supported on (e.g., movably coupled to) a second adjustable arm support (e.g., in FIG. 22, the robotic arm 210-1 is supported by the adjustable arm support 220-1 and the robotic arm 210-4 is supported by the adjustable arm support 220-2).

In some embodiments, the first robotic arm and the second robotic arm are positioned on a same side of an operating table (e.g., the robotic arms 210-1 and 210-2 in FIG. 22).

In some embodiments, the first robotic arm and the second robotic arm are supported on an adjustable arm support (e.g., in FIG. 22, the robotic arms 210-1 and 210-2 are supported on a common adjustable arm support 220-1).

In some embodiments, the surgical robotic system, regardless of the determination that the second surgical tool is within the field of view and the first surgical tool is not within the field of view, provides electrical signals for presenting the first visual indicator on the viewer.

In some embodiments, the surgical robotic system, in accordance with a determination that the first surgical tool is not within the field of view (and in some embodiments, regardless of the determination that the second surgical tool is within the field of view), provides electrical signals for presenting the first visual indicator on the viewer.

### 3. Implementing Systems and Terminology.

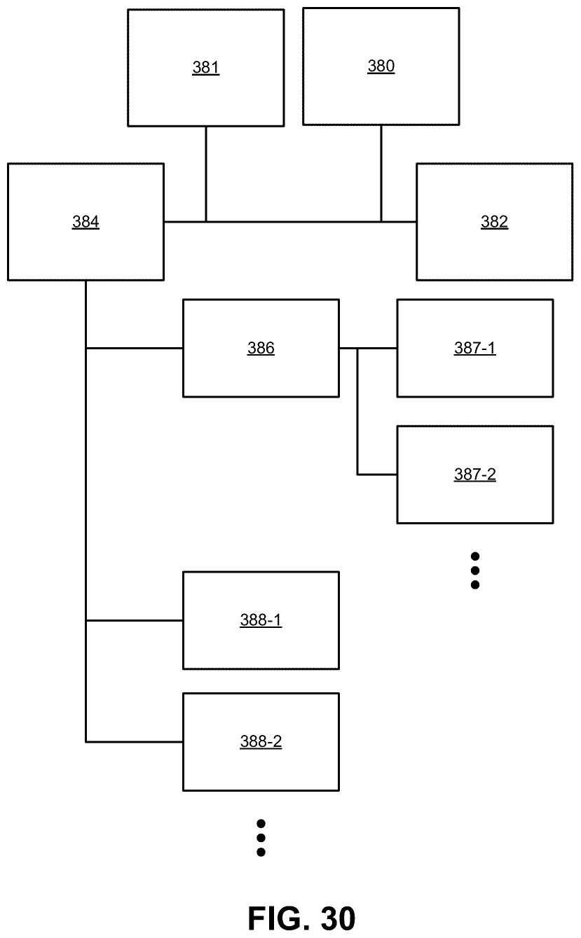

FIG. 30 is a schematic diagram illustrating electronic components of a medical robotic system (e.g., a surgical robotic system) in accordance with some embodiments.

The robotic medical system (e.g., surgical robotic system) includes one or more processors 380, which are in communication with a computer-readable storage medium 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 25, 26A, 26B, and 29A to 29C). The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controller 386 are integrated with the input/output controller 384 and the input/output controller 384 provides control signals directly to the one or more actuators 387 (without a separate actuator controller). Although FIG. 30 shows that there is one actuator controller 386 (e.g., one actuator controller for the entire medical robotic system; in some embodiments, additional actuator controllers may be used (e.g., one actuator controller for each actuator, etc.). In some embodiments, the one or more processors 380 are in communication with one or more displays 381 for displaying information as described herein.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for determining whether a tool is within or outside a surgical field of view provided by a camera or scope and rendering one or more indicators representing positions or directions of one or more medical tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the embodiments shown herein but is to be accorded the widest scope consistent with the principles and novel features disclosed herein.

### 4. Illustration of Subject Technology as Clauses.

Some embodiments or implementations are described with respect to the following clauses:

Clause 1. A robotic system, comprising:
a first robotic arm coupled to a first surgical tool;
a second robotic arm coupled to a second surgical tool;
a third robotic arm coupled to a scope;
a viewer for displaying a field of view of a surgical site derived from the scope;
one or more processors; and
memory storing instructions for execution by the one or more processors, the stored instructions including instructions for:
in accordance with a determination that the second surgical tool is within the field of view and the first surgical tool is not within the field of view, providing electrical signals for presenting a first visual indicator on the viewer, wherein the first visual indicator indicates a location of the first surgical tool.

Clause 2. The robotic system of clause 1, wherein the stored instructions also include instructions for:
providing electrical signals for presenting the first visual indicator at a first location on the viewer to indicate the location of the first surgical tool; and, subsequent to providing the electrical signals for presenting the first visual indicator at the first location on the viewer, in accordance with a determination that the location of the first surgical tool has changed, providing electrical signals for presenting the first visual indicator at a second location, on the viewer, that corresponds to the changed location of the first surgical tool.

Clause 3. The robotic system of clause 1 or clause 2, wherein the first visual indicator also indicates a distance to the first surgical tool from the field of view.

Clause 4. The robotic system of clause 3, wherein the stored instructions also include instructions for, subsequent to providing the electrical signals for presenting the first visual indicator on the viewer, in accordance with a determination that the distance from the field of view to the first surgical tool has changed, providing electrical signals for presenting an updated first visual indicator on the viewer that indicates the changed distance from the field of view to the first surgical tool.

Clause 5. The robotic system of clause 3 or clause 4, wherein the stored instructions also include instructions for, subsequent to providing the electrical signals for presenting the first visual indicator on the viewer, in accordance with a determination that the first surgical tool is within the field of view, ceasing to provide the electrical signals for presenting the first visual indicator on the viewer.

Clause 6. The robotic system of any of clauses 3-5, wherein the first visual indicator includes a graphical element having a size that indicates the distance to the first surgical tool from the field of view.

Clause 7. The robotic system of clause 6, wherein the first visual indicator includes information identifying a robotic arm associated with the graphical element.

Clause 8. The robotic system of any of clauses 1-7, wherein the first visual indicator identifies a type of the first surgical tool.

Clause 9. The robotic system of clause 8, wherein the first visual indicator includes a graphical representation of the first surgical tool.

Clause 10. The robotic system of any of clauses 1-9, wherein the first visual indicator is concurrently displayed with a display of the field of view of the surgical site on the viewer.

Clause 11. The robotic system of clause 10, wherein the first visual indicator is displayed around the display of the field of view of the surgical site.

Clause 12. The robotic system of clause 10 or clause 11, wherein the first visual indicator is displayed over the display of the field of view of the surgical site.

Clause 13. The robotic system of clause 12, wherein the stored instructions also include instructions for providing electrical signals for highlighting a predefined region of the display of the field of view of the surgical site and the first visual indicator is displayed along a periphery of the predefined region.

Clause 14. The robotic system of any of clauses 1-13, wherein the stored instructions also include instructions for providing a rendered image that includes the first visual indicator.

Clause 15. The robotic system of clause 14, wherein the rendered image also includes a second visual indicator indicating a location of the second surgical tool.

Clause 16. The robotic system of clause 15, wherein the first visual indicator includes information identifying the first robotic arm associated with the first surgical tool and the second visual indicator includes information identifying the second robotic arm associated with the second surgical tool.

Clause 17. The robotic system of clause 15 or clause 16, wherein the first visual indicator includes a graphical representation of the first surgical tool and the second visual indicator includes a graphical representation of the second surgical tool.

Clause 18. The robotic system of any of clauses 14-17, wherein the stored instructions include instructions for replacing the display of the field of view with the rendered image.

Clause 19. The robotic system of any of clauses 14-18, wherein the stored instructions include instructions for overlaying the rendered image partially over the display of the field of view.

Clause 20. The robotic system of any of clauses 1-19, wherein the stored instructions include instructions for, in response to receiving a request for displaying the first visual indicator and in accordance with the determination that second surgical tool is within the field of view and the first surgical tool is not within the field of view, providing the electrical signals for presenting the first visual indicator on the viewer.

Clause 21. The robotic system of clause 20, wherein the stored instructions include instructions for, in response to determining that the request for displaying the first visual indicator has ceased, ceasing to provide the electrical signals for presenting the first visual indicator on the viewer.

Clause 22. The robotic system of any of clauses 1-21, wherein the stored instructions include instructions for:
while the robotic system is in an indicator display mode, and in accordance with the determination that second surgical tool is within the field of view and the first surgical tool is not within the field of view, providing the electrical signals for presenting the first visual indicator on the viewer; and
while the robotic system is not in the indicator display mode, ceasing to provide the electrical signals for presenting the first visual indicator on the viewer.

Clause 23. The robotic system of clause 22, wherein the stored instructions include instructions for switching into or out of the indicator display mode in response to a user input.

Clause 24. The robotic system of any of clauses 1-23, wherein the first robotic arm and the second robotic arm are positioned bilaterally relative to an operating table.

Clause 25. The robotic system of any of clauses 1-24, wherein the first robotic arm is supported on a first adjustable arm support and the second robotic arm is supported on a second adjustable arm support.

Clause 26. The robotic system of any of clauses 1-25, wherein the first robotic arm and the second robotic arm are positioned on a same side of an operating table.

Clause 27. The robotic system of any of clauses 1-26, wherein the first robotic arm and the second robotic arm are supported on an adjustable arm support.

Clause 28. The robotic system of any of clauses 1-27, wherein the viewer is part of a surgeon console.

Clause 29. The robotic system of clause 28, wherein:
the surgeon console includes an input device; and
the stored instructions include instructions for:
receiving an input on the input device; and
in response to receiving the input on the input device, providing electrical signals for presenting a bird's eye view of the first robotic arm, the second robotic arm, and the third robotic arm.

Clause 30. The robotic system of clause 29, wherein the input device includes a foot pedal.

Clause 31. An electronic device in communication with a robotic system with a first robotic arm coupled to a first surgical tool, a second robotic arm coupled to a second surgical tool, a scope, and a viewer, the electronic device comprising:
one or more processors; and
memory storing instructions for execution by the one or more processors, the stored instructions including instructions for:
in accordance with a determination that the second surgical tool is within a field of view and the first surgical tool is not within the field of view, providing electrical signals for presenting a first visual indicator on the viewer, wherein the first visual indicator indicates a location of the first surgical tool.

Clause 32. A computer-readable storage medium storing instructions for execution by one or more processors in communication with a robotic system with a first robotic arm coupled to a first surgical tool, a second robotic arm coupled to a second surgical tool, a scope, and a viewer, the stored instructions including instructions for:
in accordance with a determination that the second surgical tool is within a field of view and the first surgical tool is not within the field of view, providing electrical signals for presenting a first visual indicator on the viewer, wherein the first visual indicator indicates a location of the first surgical tool.

Clause 33. A robotic system, comprising:
a first robotic arm coupled to a first surgical tool;
a second robotic arm coupled to a second surgical tool;
a scope;
a viewer for displaying a field of view of a surgical site derived from the scope;
one or more processors; and
memory storing instructions for execution by the one or more processors, the stored instructions including instructions for:
in response to receiving a request for displaying a first visual indicator, providing electrical signals for presenting the first visual indicator on the viewer, wherein the first visual indicator indicates a location of the first surgical tool.

Clause 34. A robotic system, comprising:
a first robotic arm coupled to a first surgical tool;
a second robotic arm coupled to a second surgical tool;
a scope;
a viewer for displaying a field of view of a surgical site derived from the scope;
one or more processors; and
memory storing instructions for execution by the one or more processors, the stored instructions including instructions for:
providing electrical signals for presenting a first visual indicator on the viewer, wherein the first visual indicator indicates a location of the first surgical tool.

Clause 35. A robotic system, comprising:
a first robotic arm coupled to a first surgical tool;
a scope;
a viewer for displaying a field of view of a surgical site derived from the scope;
one or more processors; and
memory storing instructions for execution by the one or more processors, the stored instructions including instructions for:
in response to receiving a request for displaying a first visual indicator, providing electrical signals for presenting the first visual indicator on the viewer, wherein the first visual indicator indicates a location of the first surgical tool.

Clause 36. A robotic system, comprising:
a first robotic arm coupled to a first surgical tool;
a scope;
a viewer for displaying a field of view of a surgical site derived from the scope;
one or more processors; and
memory storing instructions for execution by the one or more processors, the stored instructions including instructions for:
providing electrical signals for presenting a first visual indicator on the viewer, wherein the first visual indicator indicates a location of the first surgical tool.

What is claimed is:

1. A robotic system, comprising:
a first robotic arm coupled to a first surgical tool;
a second robotic arm coupled to a second surgical tool;
a third robotic arm coupled to a scope;
a viewer for displaying a field of view of a surgical site derived from the scope;
one or more processors; and
memory storing instructions for execution by the one or more processors, the stored instructions including instructions for:
in accordance with a determination that the second surgical tool is within the field of view and the first surgical tool is not within the field of view, providing electrical signals for presenting a first visual indicator on the viewer, wherein the first visual indicator indicates a location of the first surgical tool.

2. The robotic system of claim 1, wherein the stored instructions also include instructions for:
providing electrical signals for presenting the first visual indicator at a first location on the viewer to indicate the location of the first surgical tool; and,
subsequent to providing the electrical signals for presenting the first visual indicator at the first location on the viewer, in accordance with a determination that the location of the first surgical tool has changed, providing electrical signals for presenting the first visual indicator at a second location, on the viewer, that corresponds to the changed location of the first surgical tool.

3. The robotic system of claim 1, wherein the first visual indicator also indicates a distance to the first surgical tool from the field of view.

4. The robotic system of claim 3, wherein the stored instructions also include instructions for, subsequent to providing the electrical signals for presenting the first visual indicator on the viewer, in accordance with a determination that the distance from the field of view to the first surgical tool has changed, providing electrical signals for presenting an updated first visual indicator on the viewer that indicates the changed distance from the field of view to the first surgical tool.

5. The robotic system of claim 3, wherein the first visual indicator includes a graphical element having a size that indicates the distance to the first surgical tool from the field of view.

6. The robotic system of claim 5, wherein the first visual indicator includes information identifying a robotic arm associated with the graphical element.

7. The robotic system of claim 1, wherein the first visual indicator identifies a type of the first surgical tool.

8. The robotic system of claim 7, wherein the first visual indicator includes a graphical representation of the first surgical tool.

9. The robotic system of claim 1, wherein the first visual indicator is concurrently displayed with a display of the field of view of the surgical site on the viewer, wherein the first visual indicator is displayed around the display of the field of view of the surgical site.

10. The robotic system of claim 1, wherein the first visual indicator is concurrently displayed with a display of the field of view of the surgical site on the viewer, wherein the first visual indicator is displayed over the display of the field of view of the surgical site.

11. The robotic system of claim 10, wherein the stored instructions also include instructions for providing electrical signals for highlighting a predefined region of the display of the field of view of the surgical site and the first visual indicator is displayed along a periphery of the predefined region.

12. The robotic system of claim 1, wherein the stored instructions also include instructions for providing a rendered image that includes the first visual indicator, wherein the stored instructions include instructions for replacing the display of the field of view with the rendered image.

13. The robotic system of claim 1, wherein the stored instructions also include instructions for providing a rendered image that includes the first visual indicator, wherein the stored instructions include instructions for overlaying the rendered image partially over the display of the field of view.

14. The robotic system of claim 1, wherein the stored instructions include instructions for, in response to receiving a request for displaying the first visual indicator and in accordance with the determination that second surgical tool is within the field of view and the first surgical tool is not within the field of view, providing the electrical signals for presenting the first visual indicator on the viewer.

15. The robotic system of claim 14, wherein the stored instructions include instructions for, in response to determining that the request for displaying the first visual indicator has ceased, ceasing to provide the electrical signals for presenting the first visual indicator on the viewer.

16. The robotic system of claim 1, wherein the stored instructions include instructions for:
while the robotic system is in an indicator display mode, and in accordance with the determination that second surgical tool is within the field of view and the first surgical tool is not within the field of view, providing the electrical signals for presenting the first visual indicator on the viewer; and
while the robotic system is not in the indicator display mode, ceasing to provide the electrical signals for presenting the first visual indicator on the viewer, wherein the stored instructions include instructions for switching into or out of the indicator display mode in response to a user input.

17. The robotic system of claim 1, wherein the viewer is part of a surgeon console, wherein:
the surgeon console includes an input device; and
the stored instructions include instructions for:
receiving an input on the input device; and
in response to receiving the input on the input device, providing electrical signals for presenting a bird's eye view of the first robotic arm, the second robotic arm, and the third robotic arm.

18. The robotic system of claim 17, wherein the input device includes a foot pedal.

19. A computer-readable storage medium storing instructions for execution by one or more processors in communication with a robotic system with a first robotic arm coupled to a first surgical tool, a second robotic arm coupled to a second surgical tool, a scope, and a viewer, the stored instructions including instructions for:
in accordance with a determination that the second surgical tool is within a field of view and the first surgical tool is not within the field of view, providing electrical signals for presenting a first visual indicator on the viewer, wherein the first visual indicator indicates a location of the first surgical tool.

20. A robotic system, comprising:
a first robotic arm coupled to a first surgical tool;
a second robotic arm coupled to a second surgical tool;
a scope;
a viewer for displaying a field of view of a surgical site derived from the scope;
one or more processors; and
memory storing instructions for execution by the one or more processors, the stored instructions including instructions for:
in response to receiving a request for displaying a first visual indicator, providing electrical signals for presenting the first visual indicator on the viewer, wherein the first visual indicator indicates a location of the first surgical tool.
